(19) United States
(12) Patent Application Publication
(10) Pub. No.: US 2025/0345166 A1
(43) Pub. Date: Nov. 13, 2025

(54) COMPLIANCE DEVICE WITH ELASTIC MIDSECTION

(71) Applicant: EDWARDS LIFESCIENCES CORPORATION, Irvine, CA (US)

(72) Inventor: Ofir Witzman, Harish (IL)

(73) Assignee: EDWARDS LIFESCIENCES CORPORATION, Irvine, CA (US)

(21) Appl. No.: 19/251,562

(22) Filed: Jun. 26, 2025

Related U.S. Application Data
(63) Continuation of application No. PCT/US2023/084385, filed on Dec. 15, 2023.
(60) Provisional application No. 63/481,131, filed on Jan. 23, 2023.

(51) Int. Cl. A61F 2/07; A61F 2/24; A61L 27/54

(52) U.S. Cl. CPC A61F 2/07; A61F 2/24; A61L 27/54; A61F 2220/0008; A61F 2250/0051; A61F 2250/0069

(57) ABSTRACT

Devices, systems, and/or methods can provide compliance characteristics to fluid vessels. For example, an implant device can include an anchoring region to anchor the implant device to a blood vessel or other anatomy. The anchoring region can be disposed at one end of a flexible midsection that is configured to expand as a result of fluid flowing through the implant device. The flexible midsection can include another anchoring region at an opposite end. One or both anchoring regions are configured to encourage tissue ingrowth at an outer surface of the respective anchoring region.

### RELATED APPLICATION(S)

This application is a continuation of International Patent Application No. PCT/US2023/084385, filed Dec. 15, 2023, which claims the benefit of U.S. Provisional Patent Application Ser. No. 63/481,131, filed on Jan. 23, 2023, the complete disclosures of which are hereby incorporated by reference in their entireties.

### BACKGROUND

### Field

The present disclosure generally relates to the field of medical implant devices.

### Description of Related Art

Insufficient or reduced compliance in certain blood vessels, including arteries such as the aorta, can result in reduced perfusion, cardiac output, and other health complications. Restoring compliance and/or otherwise controlling flow in such blood vessels can improve patient outcomes.

### SUMMARY

Described herein are devices, methods, and/or systems that facilitate the restoration of compliance characteristics to undesirably stiff blood vessels and other anatomy. For example, an implant device can include proximal and distal anchoring regions or structures configured to anchor the implant device to a blood vessel, heart valve, or other anatomy. The anchoring regions or structures can be coupled to an elastic tube mid-section that is configured to expand and contract to change a volume of blood or other fluid passing therethrough. Such change in volume can allow the blood vessel to mimic compliance of a healthy blood vessel and/or otherwise promote blood flow during, for example, a phase of the cardiac cycle.

For purposes of summarizing the disclosure, certain aspects, advantages, and/or features are described. It is to be understood that not necessarily all such advantages may be achieved in accordance with any particular example. Thus, the disclosed examples can be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes. In addition, various features of different disclosed examples can be combined to form additional examples, which are part of this disclosure. Throughout the drawings, reference numbers may be reused to indicate correspondence between reference elements.

FIG. 1A illustrates an example representation of a heart and

associated vasculature having various features relevant to one or more examples of the present disclosure.

FIG. 1B illustrates an example healthy aorta.

FIG. 1C illustrates an example unhealthy aorta.

FIG. 2A provides a side view of a compliant blood vessel radially contracting/recoiling during the diastolic phase of the cardiac cycle.

FIG. 2B provides a side view of a compliant blood vessel experiencing expansion during the systolic phase of the cardiac cycle.

FIG. 2C provides a cross-sectional view of a compliant blood vessel radially contracting/recoiling during the diastolic phase of the cardiac cycle.

FIG. 2D provides a cross-sectional view of a compliant blood vessel experiencing expansion during the systolic phase of the cardiac cycle.

FIGS. 3A and 3B illustrate cross-sectional views of a blood vessel that is relatively stiff.

FIG. 4 is a graph illustrating blood pressure over time in an example healthy patient.

FIG. 5 is a graph illustrating blood pressure over time in an example patient having reduced aortic compliance.

FIG. 6A shows a perspective view of an example device that can be configured to enhance compliance of a fluid vessel, according to one or more examples.

FIGS. 6B and 6C provide side and front views, respectively, of an example device that can be configured to enhance compliance of a fluid vessel, according to one or more additional examples.

FIGS. 6D and 6E provide side and front views, respectively, of an example device that can be configured to enhance compliance of a fluid vessel, according to one or more additional examples.

FIG. 7A illustrates an example implant device with a prosthetic valve.

FIGS. 7B, 7C, and 7D illustrate example prosthetic valves that can be implemented with the implant device of FIG. 7A in accordance with one or more examples.

FIG. 8 illustrates another example prosthetic valve that can be implemented with an implant device in accordance with one or more examples.

FIG. 9 illustrates an example implant device of FIGS. 6A-6E disposed within anatomy of a patient in accordance with one or more examples.

FIGS. 10A and 10B illustrate operation of the example implant device of FIGS. 6A-6E during various phases of the cardiac cycle in accordance with one or more examples.

FIG. 11 illustrates an example implant device of FIGS. 6A-6E disposed within damaged anatomy of a patient in accordance with one or more examples.

FIGS. 12A, 12B, 12C, 12D, 12E, 12F, 12G, and 12H illustrate a flow diagram for a process for implanting an implant device in accordance with one or more examples.

FIGS. 13A, 13B, 13C, 13D, 13E, 13F, 13G, and 13H provide images of the implant device and certain anatomy corresponding to operations of the process of FIGS. 12A, 12B, 12C, 12D, 12E, 12F, 12G, and 12H according to one or more examples.

### DETAILED DESCRIPTION

The headings provided herein are for convenience and do not necessarily affect the scope or meaning of the subject matter.

Although certain examples are disclosed below, the subject matter extends beyond the specifically disclosed examples to other alternative examples and/or uses and to modifications and equivalents thereof. Thus, the scope of the claims that can arise here from is not limited by any of the examples described below. In any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain examples; however, the order of description should not be construed to imply that these operations are order dependent. Additionally, the structures, systems, and/or devices described herein can be embodied as integrated components or as separate components. For purposes of comparing various examples, certain aspects of these examples are described. Not necessarily all such aspects or advantages are achieved by any particular example. Thus, for example, various examples can be carried out in a manner that achieves or optimizes one advantage or group of advantages as taught herein without necessarily achieving other aspects or advantages as can also be taught or suggested herein.

Certain reference numbers are re-used across different figures of the figure set of the present disclosure as a matter of convenience for devices, components, systems, features, and/or modules having features that can be similar in one or more respects. However, with respect to any of the examples disclosed herein, re-use of common reference numbers in the drawings does not necessarily indicate that such features, devices, components, or modules are identical or similar. Rather, one having ordinary skill in the art can be informed by context with respect to the degree to which usage of common reference numbers can imply similarity between referenced subject matter. Use of a particular reference number in the context of the description of a particular figure can relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to the numeric portion (e.g., ‘10’) can refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present disclosure to a feature ‘10’ can be refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, these terms are used herein for ease of description to describe the positional relationship between element(s)/structures(s), as illustrated in the drawings. Spatially relative terms are generally intended to encompass different orientations of the element(s)/structures(s), in use or operation, in addition to the orientations depicted in the drawings. For example, an element/structure described as “above” another element/structure can represent a position that is below or beside such other element/structure with respect to alternate orientations of the subject patient or element/structure, and vice-versa. Spatially relative terms, including those listed above, can be relative to a respective illustrated orientation of a referenced figure.

Any of the example methods and/or structures disclosed herein for treating a patient also encompass analogous methods and/or structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies. The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), computer simulator, imaginary person, imaginary anatomy, etc.

Any of the various systems, devices, apparatuses, etc. in this disclosure can be sterilized (e.g., with heat, radiation, ethylene oxide, hydrogen peroxide, etc.) to ensure they are safe for use with patients, and/or the methods herein can comprise sterilization of the associated system, device, apparatus, etc. (e.g., with heat, radiation, ethylene oxide, hydrogen peroxide, etc.).

### Vascular Anatomy and Compliance

Certain examples are disclosed herein in the context of vascular implant devices, and in particular, compliance-enhancement implant devices implanted in the aorta. However, although certain principles disclosed herein can be particularly applicable to the anatomy of the aorta, the compliance-enhancement implant devices in accordance with the present disclosure can be implanted in, or configured for implantation in, any suitable or desirable blood vessels or other anatomy, such as the inferior vena cava, etc.

The anatomy of the heart and vascular system is described below to assist in the understanding of certain concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves can be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles can be prompted by signals generated by the electrical system of the heart.

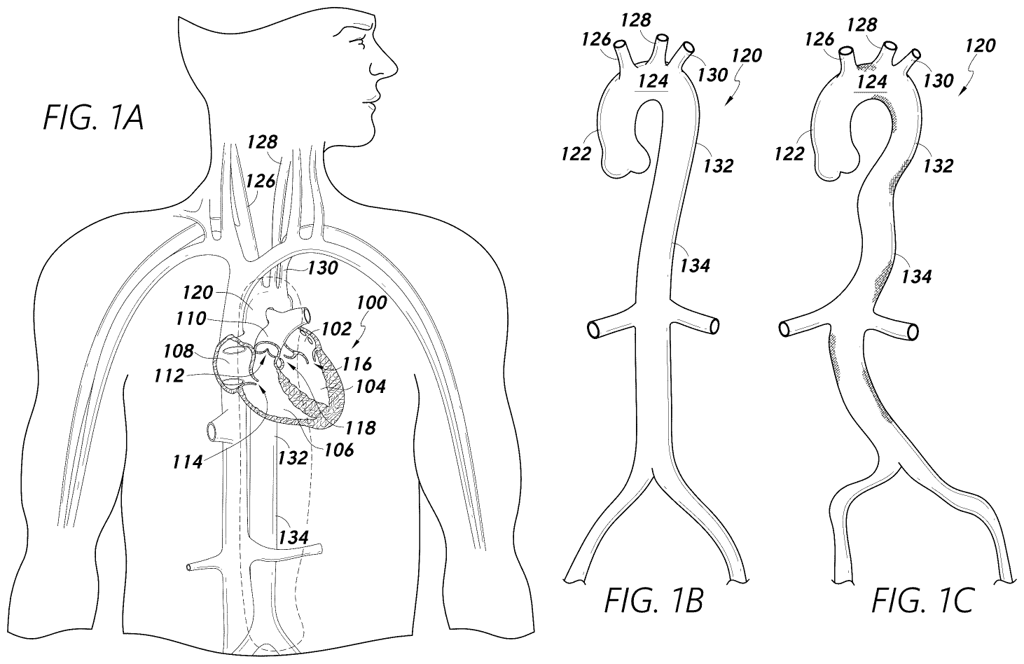

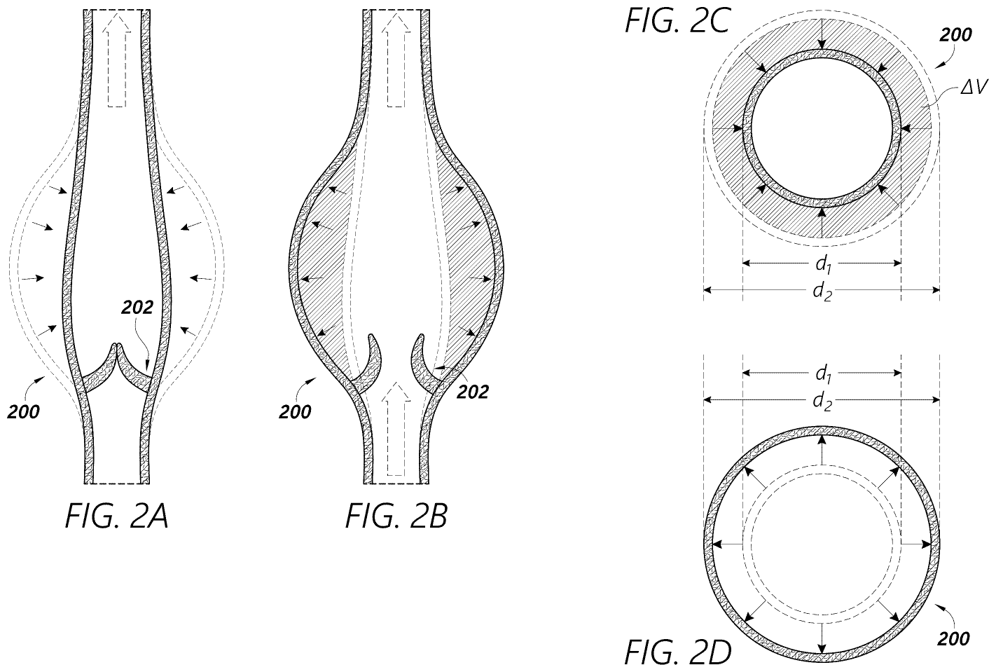

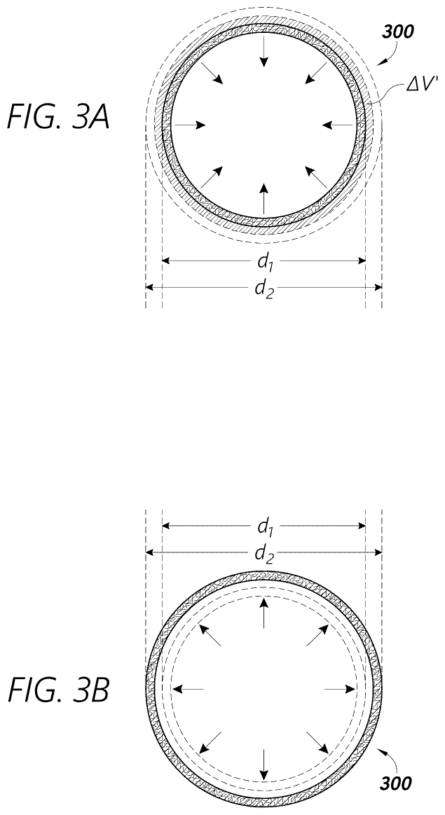

FIG. 1A illustrates an example representation of a heart 100 and associated vasculature having various features relevant to one or more examples of the present disclosure. The heart 100 includes four chambers, namely the left atrium 102, the left ventricle 104, the right ventricle 106, and the right atrium 108. In terms of blood flow, blood generally flows from the right ventricle 106 into the pulmonary artery 110 via the pulmonary valve 112, which separates the right ventricle 106 from the pulmonary artery 110 and is configured to open during systole so that blood can be pumped toward the lungs and close during diastole to prevent blood from leaking back into the heart from the pulmonary artery 110. The pulmonary artery 110 carries deoxygenated blood from the right side of the heart 100 to the lungs. The pulmonary artery 110 includes a pulmonary trunk and left and right pulmonary arteries that branch off of the pulmonary trunk, as shown.

The tricuspid valve 114 separates the right atrium 108 from the right ventricle 106. The tricuspid valve 114 generally has three cusps/leaflets and can generally close during ventricular contraction (i.e., systole) and open during ventricular expansion (i.e., diastole). The mitral valve 116 generally has two cusps/leaflets and separates the left atrium 102 from the left ventricle 104. The mitral valve 116 is configured to open during diastole so that blood in the left atrium 102 can flow into the left ventricle 104, and, when functioning properly, closes during systole to prevent blood from leaking back into the left atrium 102. The aortic valve 118 separates the left ventricle 104 from the aorta 120. The aortic valve 118 is configured to open during systole to allow blood leaving the left ventricle 104 to enter the aorta 120, and close during diastole to prevent blood from leaking back into the left ventricle 104.

The heart valves can generally comprise a relatively dense fibrous ring, referred to herein as the annulus, as well as a plurality of leaflets or cusps attached to the annulus. Generally, the size of the leaflets or cusps can be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel can become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (mitral and tricuspid) heart valves generally are coupled to a collection of chordae tendineae and papillary muscles (not shown for visual clarity) for securing the leaflets of the respective valves to promote and/or facilitate proper coaptation of the valve leaflets and prevent prolapse thereof. The papillary muscles, for example, can generally comprise finger-like projections from the ventricle wall. The valve leaflets are connected to the papillary muscles by the chordae tendineae. A wall of muscle, referred to as the septum, separates the left 102 and right 108 atria and the left 104 and right 106 ventricles.

The vasculature of the human body, which can be referred to as the circulatory system, cardiovascular system, or vascular system, contains a complex network of blood vessels with various structures and functions and includes various veins (venous system) and arteries (arterial system). Generally, arteries, such as the aorta, carry blood away from the heart, whereas veins, such as the inferior and superior vena cavae, carry blood back to the heart.

FIGS. 1B and 1C show detailed views of example healthy and aged/stiff aortas 120, respectively. The aorta 120 is a compliant arterial blood vessel that buffers and conducts pulsatile left ventricular output and contributes the largest component of total compliance of the arterial tree. The aorta 120 includes the ascending aorta 122, which begins at the opening of the aortic valve 118 in the left ventricle 104 of the heart 100. The ascending aorta 122 and pulmonary trunk 110 twist around each other, causing the aorta 120 to start out posterior to the pulmonary trunk 110, but end by twisting to its right and anterior side. Among the various segments of the aorta 120, the ascending aorta 122 is relatively more frequently affected by aneurysms and dissections, often requiring open heart surgery to be repaired. The transition from ascending aorta 122 to aortic arch 124 is at the pericardial reflection on the aorta 120. At the root of the ascending aorta 122, the lumen has three small pockets between the cusps of the aortic valve 118 and the wall of the aorta 120, which are called the aortic sinuses or the sinuses of Valsalva. The left aortic sinus contains the origin of the left coronary artery and the right aortic sinus likewise gives rise to the right coronary artery. Together, these two arteries supply the heart with blood.

As mentioned above, the aorta 120 is coupled to the heart 100 via the aortic valve 118, which leads into the ascending aorta 122 and gives rise to the innominate artery 126, the left common carotid artery 128, and the left subclavian artery 130 along the aortic arch 124 before continuing as the descending thoracic aorta 132 and further the abdominal aorta 134. References herein to the aorta can be understood to refer to the ascending aorta 122 (also referred to as the “ascending thoracic aorta”), aortic arch 124, descending or thoracic aorta 132 (also referred to as the “descending thoracic aorta”), abdominal aorta 134, or other arterial blood vessel or portion thereof.

Arteries, such as the aorta 120, can utilize blood vessel compliance (e.g., arterial compliance) to store and release energy through the stretching of blood vessel walls. The term “compliance” can be used herein according to its broad and ordinary meaning, and can refer to the ability of an arterial blood vessel or prosthetic implant device, or portion thereof, to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

Arterial compliance facilitates perfusion of organs in the body with oxygenated blood from the heart. Generally, a healthy aorta and other major arteries in the body are at least partially elastic and compliant, such that they can act as a reservoir for blood, filling up with blood when the heart contracts during systole and continuing to generate pressure and push blood to the organs of the body during diastole. In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood can flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

A healthy aorta, as shown in FIG. 1B, runs along a generally straight path, whereas an aged and/or stiffened aorta, as shown in FIG. 1C, can run along a more tortuous, curved path. That is, the aorta tends to change in shape as a function of age, resulting in higher degrees of curvature or tortuosity, as developed gradually over time. Such change in shape of the blood vessel can be associated with the vasculature of the subject becoming less elastic. As such conditions develop, arterial blood pressure (e.g., left-ventricular afterload) can become more pulsatile, which can have deleterious effects, such as the thickening of the left ventricle (LV) muscle, and insufficient perfusion of the heart. Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles, and peripheral edema, for example, which may be caused by structural and/or functional cardiac abnormality. Such conditions can result in reduced cardiac output and/or elevated intra-cardiac pressures at rest or during stress.

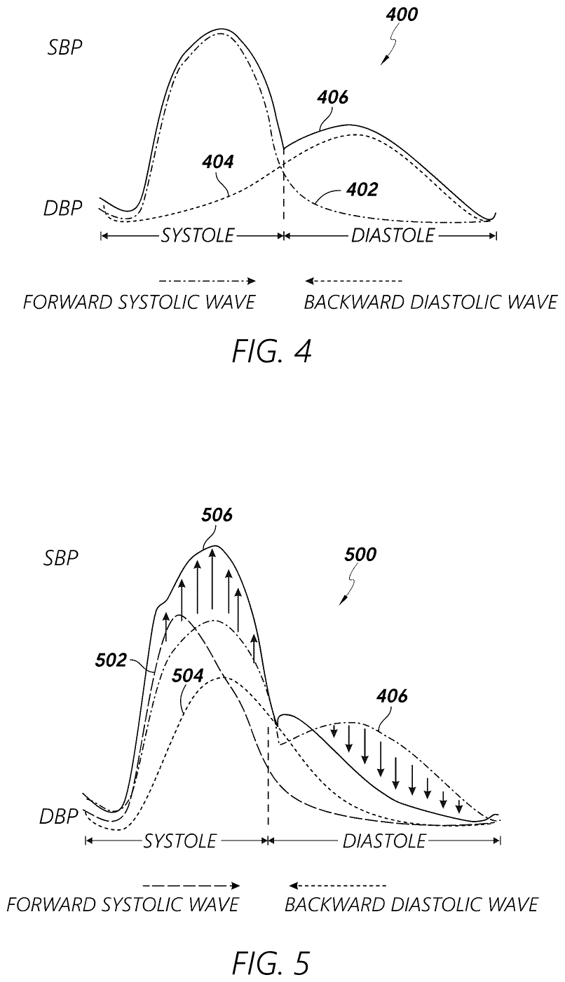

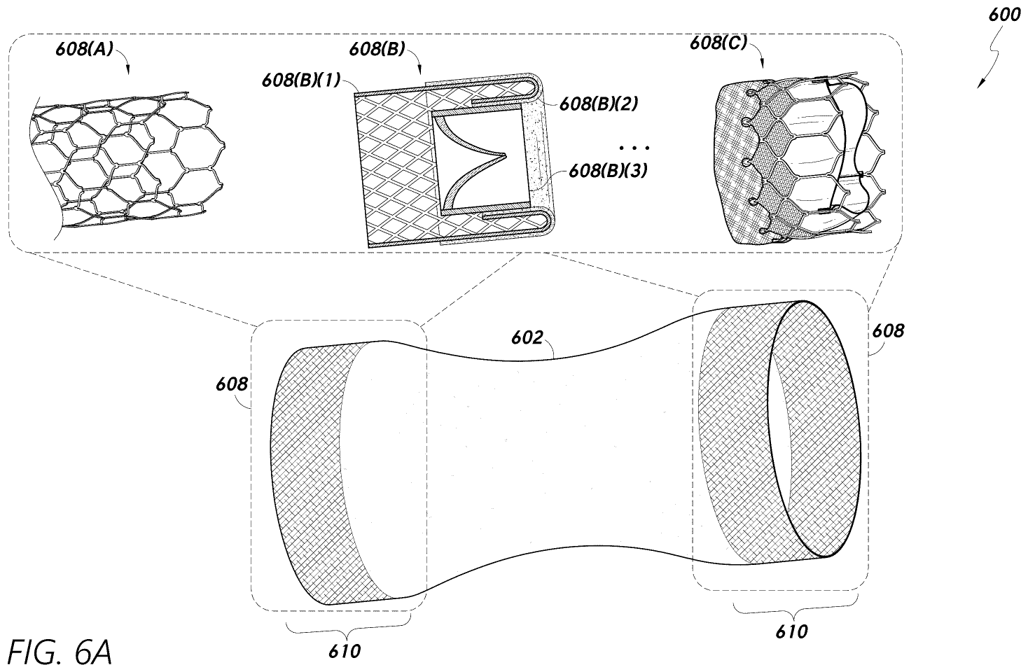

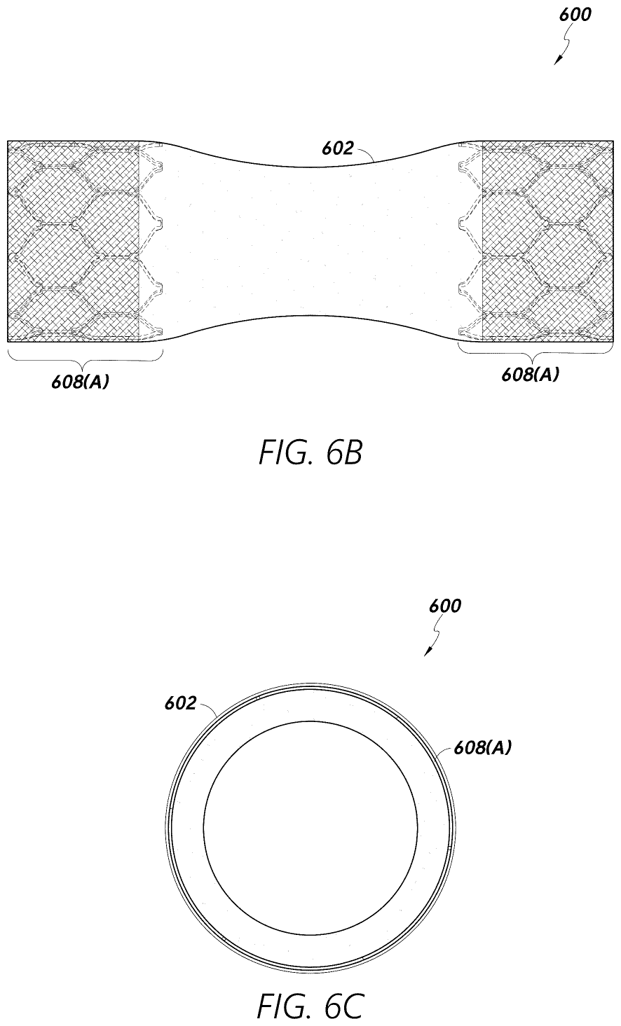

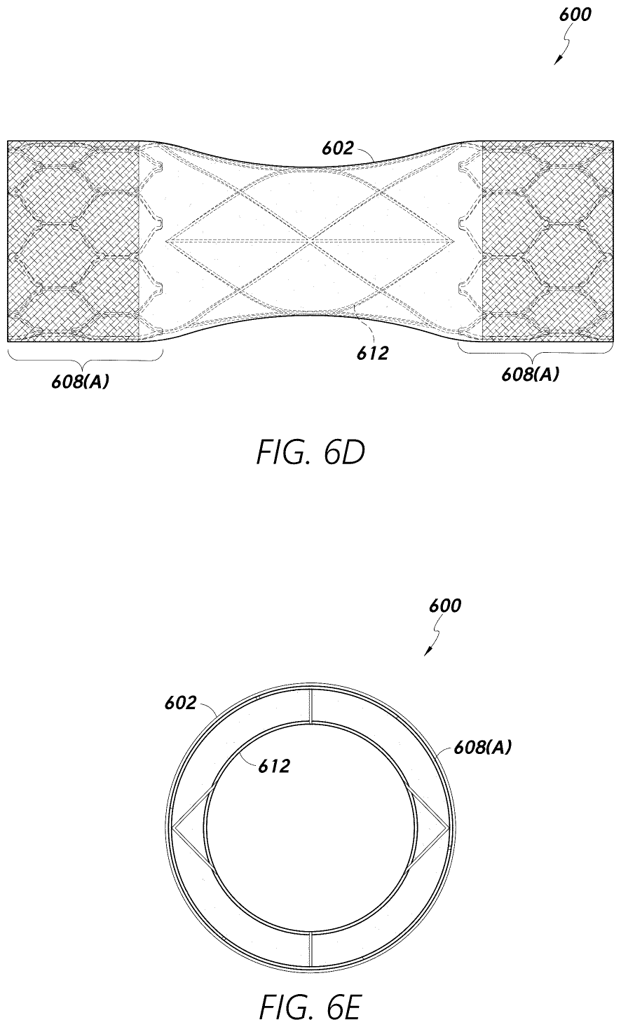

FIGS. 2A and 2C provide side and cross-sectional views, respectively, of a compliant blood vessel 200, such as an artery (e.g., aorta), radially contracting/recoiling during the diastolic phase of the cardiac cycle. FIGS. 2B and 2D provide side and cross-sectional views, respectively, of the compliant blood vessel 200 experiencing expansion during the systolic phase of the cardiac cycle. As understood by those having ordinary skill in the art, the systolic phase of the cardiac cycle is associated with the pumping phase of the left ventricle, while the diastolic phase of the cardiac cycle is associated with the filling phase of the left ventricle. As identified in FIG. 2C, with proper arterial compliance, a change in volume 4V will generally occur in an artery between high- and low-pressure phases of the cardiac cycle. With respect to the aorta 200, as shown in FIGS. 2B and 2D, as blood is pumped into the aorta 200 through the aortic valve 202, the pressure in the aorta 200 increases and the diameter of at least a portion of the aorta 200 expands. A first portion of the blood entering the aorta 200 during systole may pass through the aorta 200 during the systolic phase, while a second portion (e.g., approximately half of the total blood volume) may be stored in the expanded volume ΔV caused by compliant stretching of the blood vessel, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance can have a significant effect on perfusion and/or blood pressure in some patients. For example, arteries with relatively higher compliance can be conditioned to more easily deform than lower-compliance arteries under the same pressure conditions. Compliance (C) can be calculated using the following equation, where ΔV is the change in volume (e.g., in mL) of the blood vessel, and ΔP is the pulse pressure from systole to diastole (e.g., in mmHg):

$$C = \frac{Δ V}{Δ P} \qquad ( 1 )$$

Aortic stiffness and reduced or diminished compliance can lead to elevated systolic blood pressure, which can in turn lead to elevated intracardiac pressures, increased afterload, and/or other complications that can exacerbate heart failure. Aortic stiffness further can lead to reduced diastolic flow, which can lead to reduced coronary perfusion, decreased cardiac supply, and/or other complications that can likewise exacerbate heart failure.

Arterial compliance restoration devices, methods, and concepts disclosed herein may be generally described in the context of the aorta 120. However, such devices, methods and/or concepts can be applicable in connection with any other artery or blood vessel.

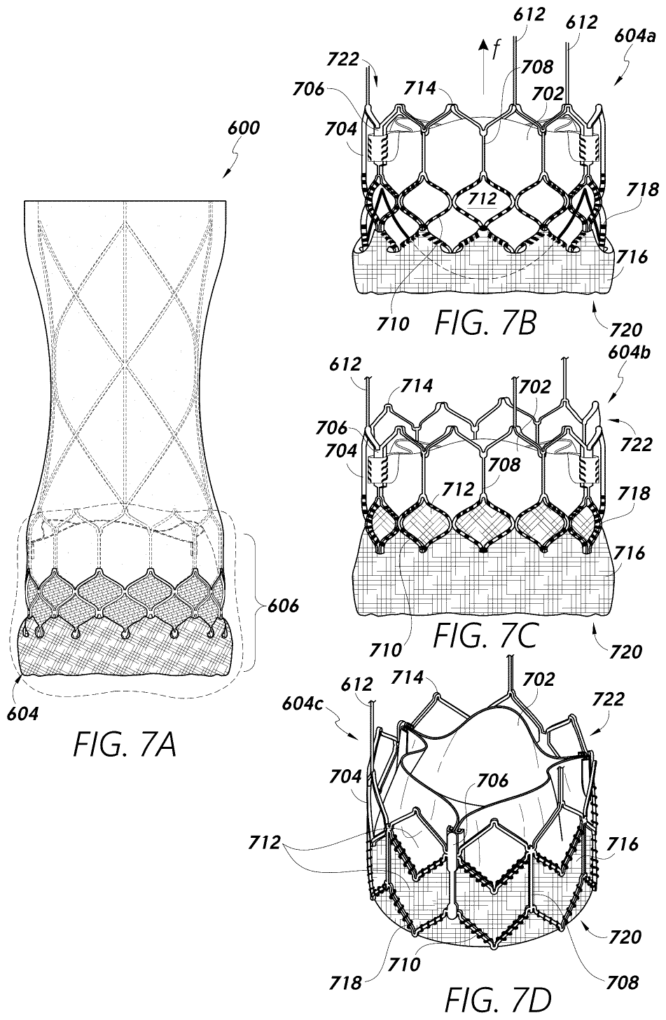

FIGS. 3A and 3B show a cross-sectional profile of a blood vessel 300 that is relatively stiff, such as the blood vessel shown in FIG. 1C, wherein the compliance of the vessel portion 300 is diminished relative to the healthy aorta as shown in FIG. 1B. Due to the stiffness of the blood vessel wall, the blood vessel 300 can expand a relatively limited amount between diastole (shown in FIG. 3A) and systole (shown in FIG. 3B). That is, during systole, the increased fluid pressure within the blood vessel 300 can result in a relatively small and/or negligible expansion of the diameter of the blood vessel 300, as shown with respect to the difference between the contracted diameter d₁ and the expanded diameter d₂. Due to the limited expansion of the blood vessel 300, the change in volume ΔV′ in the blood vessel between phases of the cardiac cycle can likewise be limited, and therefore relatively little energy is stored in the blood vessel wall and returned to the blood circulation during low-pressure conditions, resulting in more pulsatile blood flow compared to healthy, compliant aortic tissue.

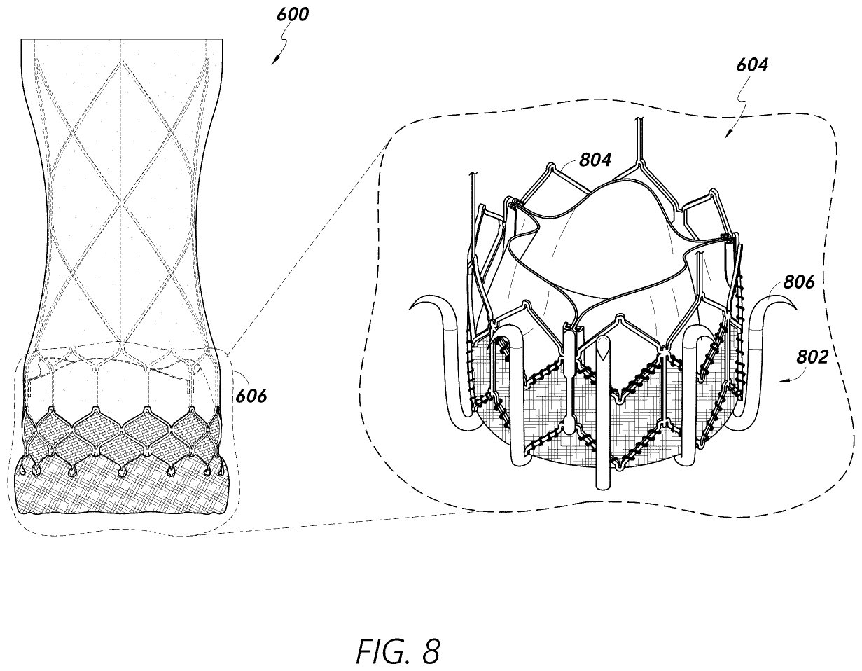

FIG. 4 is a graph 400 illustrating blood pressure over time in an example patient with a healthy, compliant aorta, wherein arterial blood pressure is represented as a combination of a forward systolic pressure wave 402 and a backward diastolic pressure wave 404. The combination of the systolic wave 402 and the diastolic wave 404 is represented by the waveform 406.

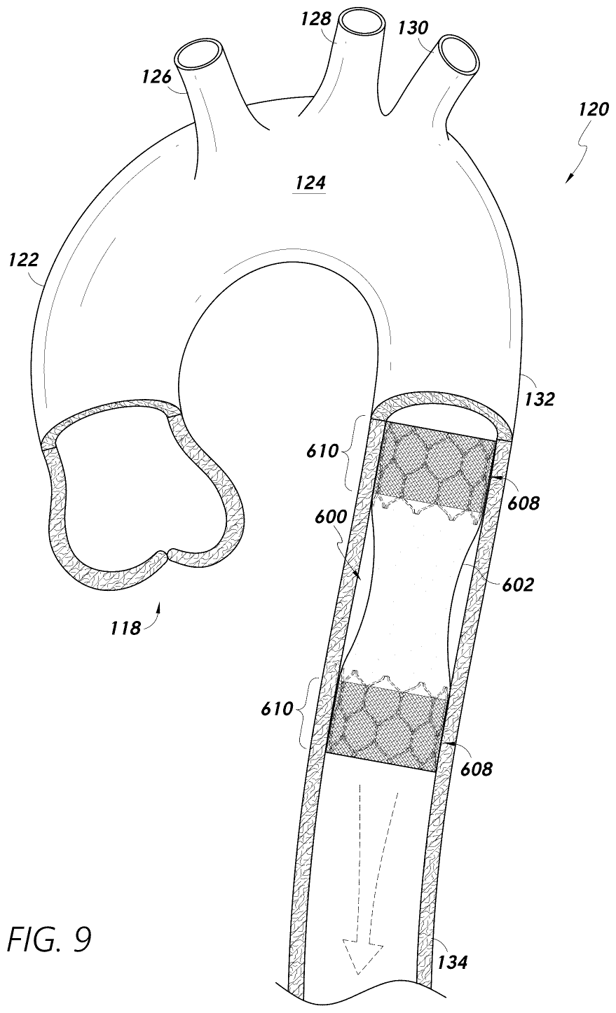

FIG. 5 is a graph 500 illustrating blood pressure over time in an example patient having reduced aortic compliance. The graph 500 shows, for reference purposes, the example combined wave 406 shown in FIG. 4. When low compliance is exhibited, less energy can be stored in the aorta compared to a healthy patient. Therefore, the systolic waveform 502 can demonstrate increased pressure during the systolic phase relative to a patient having normal compliance, while the diastolic waveform 504 can demonstrate reduced pressure during the diastolic phase relative to a patient having normal compliance. Therefore, the resulting combined waveform 506 can represent an increase in the systolic peak and a drop in the diastolic pressure, which can cause various health complications. For example, the change in waveform can impact the workload on the left ventricle and can adversely affect coronary profusion.

In view of the health complications that can be associated with reduced arterial compliance, as described above, it can be desirable in certain patients and/or under certain conditions, to at least partially alter compliance properties of the aorta or other artery or blood vessel, or otherwise alter/control flow therein, in order to improve cardiac and/or other organ health.

### Compliance-Enhancing Devices

The present disclosure relates to systems, devices, and methods for at least partially increasing and/or restoring compliance to a fluid vessel, such as the aorta 120 or other arterial (or venous) blood vessel(s), to provide improved perfusion of the heart muscle and/or other organ(s) of the body. Examples of the present disclosure can include compliant tubular devices configured to channel blood circulation therethrough, such that elastic expansion of the tube during systole can be returned to the circulation during diastole to thereby reduce systolic pressure and/or increase diastolic pressure. For instance, a compliance device in accordance with the present disclosure can include an expandable/elastic fluid channel that expands and stores energy during higher-pressure periods of the cardiac cycle (e.g., during the systolic phase) and contracts/compresses during lower-pressure period (e.g., during the diastolic phase) to return the stored energy to the circulation and increase flow through the channel.

In examples, devices of the present disclosure include elastic/compliant tubes configured to couple to an interior wall of a blood vessel such as the aorta 120. The device can comprise a tubular graft, or the like, having proximal and distal anchoring regions and a flexible tubular mid-section disposed there between. For instance, the proximal end and the distal end of the device can each include an anchoring feature configured to secure the device to the tissue of an interior portion of an area within the associated blood vessel. The flexible mid-section can be floating or unattached to tissue within the blood vessel. In some embodiments, one or both ends of the device can include additional anchoring or functional features, such as an extendable stent, one or more pins, spikes or other anchoring components, a prosthetic heart valve, or other like features. The device can allow blood to flow through the tubular graft and to the native blood vessels. The flexible mid-section of the tubular graft can be configured to expand and contract with the cardiac cycle. Thus, the device can increase a compliance of the blood vessel.

In examples, by disposing compliant implants within native anatomy/blood vessel, incidences of blood leakage and/or rupture of the expandable inner tube may be contained within the target blood vessel, thereby reducing hazards associated with extravascular arterial blood leakage, such as within the abdominal and/or chest cavity. Further, in examples, the compliant devices can be delivered through a minimally invasive procedure, which can help prevent complications associated with other types of procedures.

Methods and/or structures disclosed herein for treating a patient also encompass analogous methods and/or structures performed on or placed on a simulated patient, which is useful, for example, for training, for demonstration, for procedure and/or device development, and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof, synthetic, or any combination of natural and synthetic. Virtual elements can be entirely in silica or overlaid on one or more of the physical components. Virtual elements can be presented on any combination of screens, headsets, holographs, projections, loudspeakers, headphones, pressure transducers, temperature transducers, or using any combination of suitable technologies.

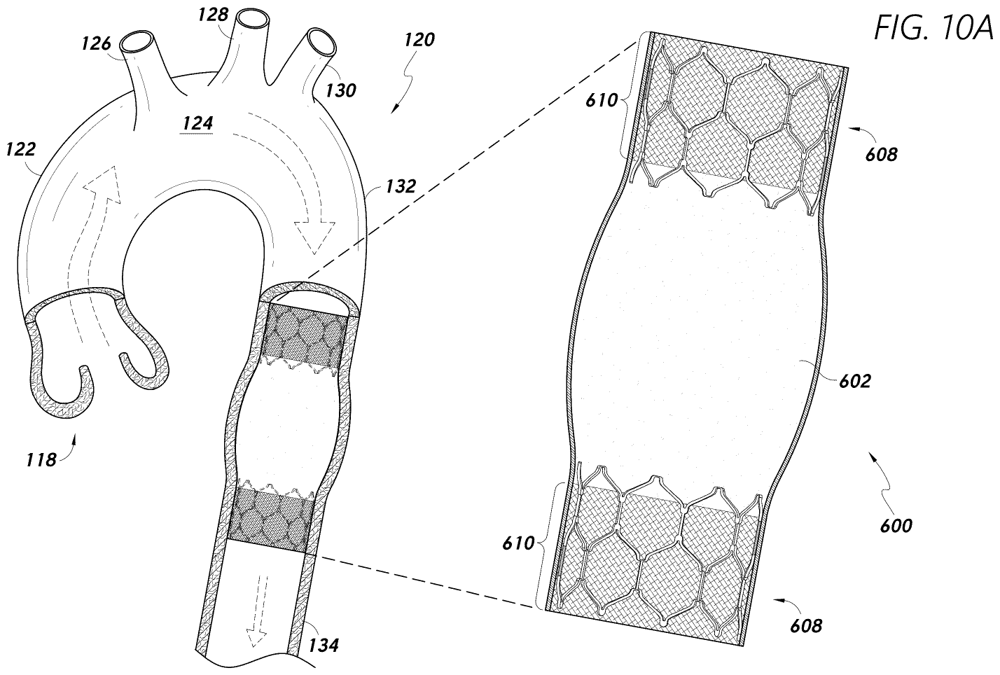

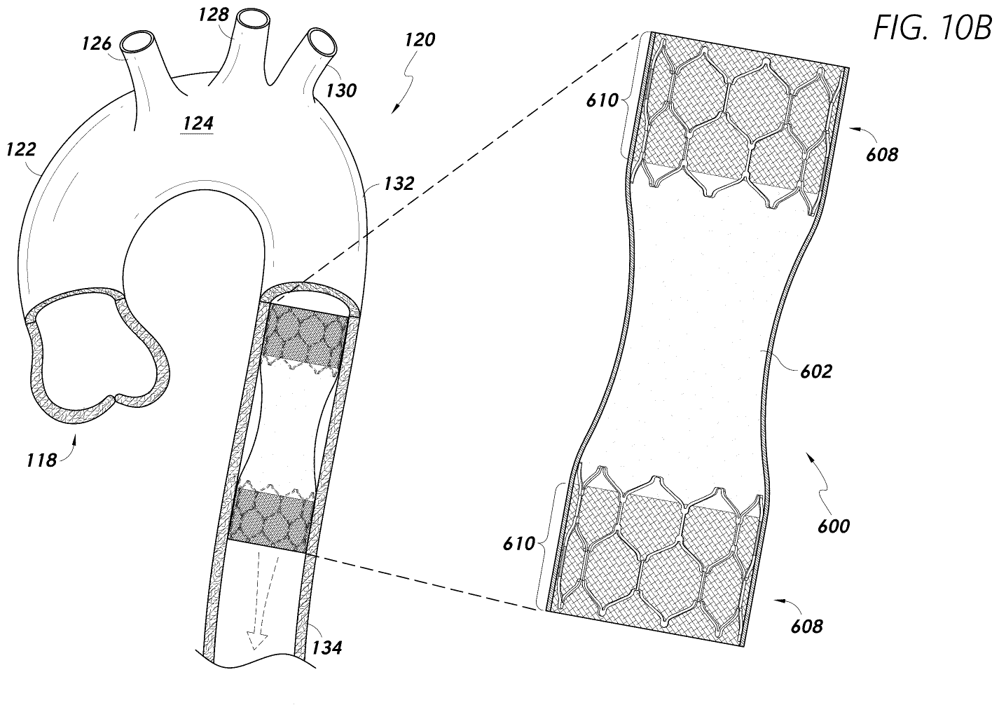

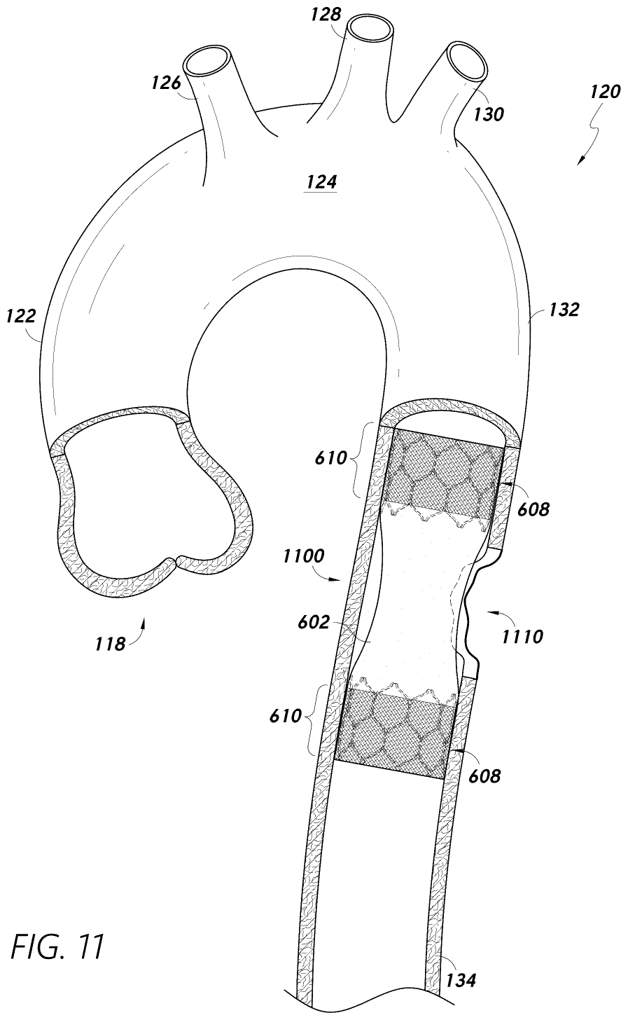

FIG. 6A illustrates a perspective view of an example device/system 600 (also referred to as “the implant device 600” or “the device 600”) that can be configured to enhance compliance of a fluid vessel. The device 600 is an implantable pliant/elastic/compliant/flexible/expandable tube/channel/conduit device. The tube-like device 600 has first and second ends, where each end comprises an anchoring region 610. A flexible midsection 602 is disposed between the first and second anchoring regions 610. The midsection 602 is configured to radially expand and/or contract, such as based on luminal/radial pressure (e.g., fluid pressure) within the device 600.

The first and second anchoring regions 610 are configured to anchor the implant device 600 within an anatomical structure, such as within a segment of a blood vessel, or the like. The first and second anchoring regions 610 may be comprised of a different material than a remainder of the device 600, which can allow or enhance the anchoring capabilities of the anchoring regions 610. For example, the material of the first and second anchoring regions 610 can be configured to encourage tissue ingrowth at outer surfaces of the first and second anchoring regions 610. The device 600 can also include a feature/structure/element(s) 608 associated with (e.g., coupled to or integral with) one or both of the anchoring regions 610 of the device 600. The feature 608 (also referred to as “the anchoring feature 608” or “the anchoring structure 608”) can be configured to anchor/secure/attach the device 600 to the target anatomy (e.g., a wall of a target blood vessel or other anatomy) and/or may be configured to facilitate various other functionality.

The feature 608 can comprise one or more of various anchoring or securing materials/components/systems, which may be disposed at each end of the device 600 or which may be disposed at a single end. In other words, the same feature 608 or a different feature 608 can be used at each end of the device 600 as desired. In one example, the feature 608 can include a stent or stent-like frame (as shown at 608(A), for example). In another example, the feature 608 can comprise a prosthetic valve, such as a prosthetic heart valve (as shown at 608(C), for example). In a further example, the feature 608 can comprise a “docking station” for a prosthetic heart valve or other bio-compatible and implantable component (as shown at 608(B), for example). In other examples, the feature 608 can include various other materials, components, or systems, including pins, spikes, anchors, sutures, and so forth, which may be disposed primarily on the outer surfaces of the proximal and distal anchoring regions 610 so as to adhere to the inner surface of the blood vessel.

The device 600 can further include a frame-like form 612 that is generally disposed within the midsection 602 and configured to maintain the tube-like shape of the midsection 602. For instance, the form 612 can help to prevent the midsection 602 from radially compressing/collapsing.

Referring to FIGS. 6A and 6B, the midsection 602 (sometimes referred to as “the radially-expandable tube 602”) can be constructed of a pliant material that is liquid-tight, such as an elastomeric polymer or other material configured to radially expand/stretch and contract/recoil in response to changing pressure/force conditions. For instance, the midsection 602 can include a thermoplastic polyurethane (TPU), nylon, etc. In some examples, the midsection 602 comprises biological tissue. Further, in some examples, the midsection 602 comprises a woven structure, such as a woven memory metal braided structure, or the like. The flexible material of the midsection 602 may be under some tension in the relaxed state, or may be without tension, depending on the material selected and the construction, as desired for closely imitating the expansion and contraction of a healthy blood vessel such as the aorta.

The midsection 602 can take a cylindrical shape/form or another shape/form when in a non-expanded/default state. That is, the midsection 602, in a natural, relaxed, and/or de-pressurized configuration/state can have a straight cylindrical shape/form. In some instances, in a radially-expanded state (pressurized configuration/state), the midsection 602 can have an at least partly outwardly-/externally-convex cylindrical shape. The midsection 602 can be configured to cycle/change between a relaxed state in low-pressure periods (e.g., diastolic phase of the cardiac cycle), wherein the midsection 602 is generally cylindrical or slightly concave or convex in the relaxed state, and a radially expanded state in high-pressure periods (e.g., systolic phase of the cardiac cycle), wherein the midsection 602 radially expands/bows-outward. The midsection 602 can function as an arterial/blood flow optimizer to generate vascular compliance. The elastic contraction of the midsection 602 during a pressure decrease within a vessel can assist in moving fluid through the vessel.

In various embodiments, the midsection 602 is fabricated from one or more compounds that discourage/inhibit/prevent/impede/obstruct tissue ingrowth. Such a compound (e.g., a thromboresistant compound) can include components that inhibit protein and cell accumulation, thrombin and fibrin formation, and platelet activation and collection. For example, the midsection 602 can be comprised of a thromboresistant material to mitigate any tissue ingrowth between the midsection 602 and the wall of the blood vessel (or other target anatomy). Alternately, the midsection 602 can be coated with one or more coatings of a thromboresistant material. Such a coating may include covering an outer surface of the midsection 602, and can also include coating an inner surface of the midsection 602. The occurrence of tissue buildup on (or within) the midsection 602 can reduce its elasticity and pliant characteristics. Thus, tissue growth can reduce the effectiveness of the midsection 602 to enhance vascular compliance.

The anchoring regions 610 can be implemented in a variety of manners to anchor/secure/attach/adhere the device 600 to the target tissue and/or to provide other functionality. For example, the anchoring regions 610 can anchor the device 600 within a native blood vessel, or other anatomical tissue. In examples, the anchoring regions 610 can anchor the device 600 to a portion of blood vessel in one of various parts of the patient anatomy, to a portion of blood vessel within proximity to the heart (e.g., within a predetermined distance), and/or to a portion of a heart chamber within proximity to the heart (e.g., within a predetermined distance).

In examples, the anchoring regions 610 can be implemented as a fabric configured to couple to the target anatomy. The fabric, which can comprise a natural or synthetic fabric or tissue (or a combination) can be configured to adhere to the inner wall of a blood vessel, for instance. The anchoring regions 610 can be fabricated from a pliant material, which is capable of conforming to the shape and flexibility of the native blood vessel. The anchoring regions 610 can be fabricated from a thermoplastic polyurethane (TPU), nylon, a biological tissue, etc. In some examples, the anchoring regions 610 comprise a woven structure, such as a woven textile or memory metal braided structure, or the like.

The anchoring regions 610 can be designed to encourage tissue ingrowth along their outer surface, so as to enhance attachment to the tissue of the interior of the blood vessel. The fabric may be coated with one or more coatings or be fabricated using one or more compounds that encourages tissue ingrowth. For instance, such a compound can include components that promote protein and cell accumulation, thrombin and fibrin formation, and platelet activation and collection. The collection of tissue between the outer surface of the anchoring regions 610 and the interior wall of the target vessel can assist in sealing the device 600, and particularly the anchoring regions 610 of the device 600 to the interior of the target blood vessel (or other anatomy), In an example, the inner surface of one or both of the anchoring regions 610 is free from a compound that encourages tissue ingrowth. Alternately, inner surfaces of the anchoring region(s) 610 can be coated with one or more coatings of a thromboresistant material, which can inhibit tissue buildup at the inner surfaces. The occurrence of excessive tissue buildup within the anchoring regions 610 may lead to a reduction in the flow of blood through the implant device 600.

The anchoring regions 610 can attach themselves to tissue by virtue of the material of the anchoring regions 610 and/or over time by virtue of the collection of ingrowth tissue between the anchoring regions 610 and the tissue of the interior wall of the vessel. Alternately or additionally, the anchoring regions 610 can be manipulated by a device/physician to attach to the tissue (e.g., device/physician attachments). In examples, the device 600 can be configured to be compressed (e.g., radially compressed to a delivery/compressed state) and transported within a delivery catheter/sheath or other tubular delivery system, such as in the case of a minimally invasive procedure through a percutaneous opening or natural orifice. As such, the device 600 can be a percutaneously-placeable implant. Alternatively, the device 600 can be implanted through another type of procedure, such as an open surgery.

One or both of the anchoring regions 610 can include one or more added anchoring features 608 in various examples. An anchoring feature 608 can be implemented in a variety of manners to additionally/alternately anchor/secure/attach the device 600 to the target tissue and/or to provide other functionality. For example, the anchoring feature 608 can anchor the device 600 within the native blood vessel, or other anatomical tissue. In examples, the anchoring feature 608 can include a device or system configured to anchor the device 600 to a portion of blood vessel within proximity to the heart (e.g., within a predetermined distance), and/or portion of a heart chamber within proximity to the heart (e.g., within a predetermined distance).

In examples, the anchoring feature 608 can be implemented as elements that are self-expandable, balloon/device expandable, or otherwise configured to expand or deform to couple to the anatomy. For instance, the anchoring feature 608 can be attached to tissue in a self-expanding/contracting manner. Alternatively, or additionally, the anchoring feature 608 can be manipulated by a device/physician to attach to the tissue (e.g., device/physician expandable/attachable). In examples, the anchoring features 608 of the device 600 can also be configured to be compressed (e.g., radially compressed to a delivery/compressed state) for transport and delivery within a delivery catheter/sheath or other tubular delivery system, such as in the case of a minimally invasive procedure through a percutaneous opening or natural orifice. As such, the anchoring features 608 can be a percutaneously-placeable or otherwise surgically implanted as components of the device 600.

In some instances, the anchoring feature 608 include barbs, patches, pins, coils, screws, tabs, hooks, wires, spikes, or other tissue anchor means configured to embed in and/or hold to the associated anatomy. For example, the anchoring feature 608 can have wires or barbs having free ends that project radially outward to puncture the tissue of the native anatomy and secure the device 600 in place. In some implementations, such wires/barbs have shape-memory that predisposes such structure to deflect radially outwardly once deployed from a capsule/sheath to facilitate anchoring of the device 600 to the native blood vessel. The anchoring regions 610 can include any number of anchoring features 608 disposed at the device 600.

As shown in FIG. 6A, in an example the anchoring feature 608 is implemented as/with a stent/frame structure 608(A). The frame 608(A) can have a structure comprising a plurality of struts that may form an array of cells, which can have any suitable or desirable shape (e.g., oval/ellipse, diamond/rhombus, hexagonal diamond/polygon, etc.). The cells can be arranged in any number of columns in the circumferential direction and/or rows in the axial, or lengthwise, direction.

The frame 608(A) can be formed using any suitable process, such as by stamping or machining the frame structure from a sheet or tube of metal. The frame 608(A) can be made of an at least partially rigid material, such as metal, plastic, etc. For example, the frame 608(A) can comprise stainless steel or nitinol. Where nitinol or other shape-memory metal or material is implemented, the frame 608(A) can be self-expanding. For instance, an expandable stent-type frame 608(A) can be configured to expand radially from a compressed delivery configuration to the expanded state shown at 608(A). In some implementations, an array of struts is formed from a sheet of metal, which is rolled into a cylinder to form the tubular/cylindrical form of the frame 608(A). In some implementations, a balloon catheter is used to deliver and/or expand the frame 608(A) for securing in a blood vessel, or other anatomy.

Referring to the side view at FIG. 6B and the end view at FIG. 6C, in some cases, the outside or inside of the frame 608(A) is covered with a fabric, polymer cover, or other material. The cover can be disposed on an outer surface or area of the frame 608(A), and/or can be disposed/applied to the inner diameter of the frame 608(A) on an inside thereof. In some implementations, the cover comprises a cloth or polymer sleeve which can be at least partially elastic, or alternatively nonelastic. The cover can be applied over or within the frame 608(A) in any suitable or desirable manner. For example, the cover can be applied using an electrical or mechanical spinning (e.g., rotary jet spinning, electrospinning, or similar) application process or other deposition process. The cover can promote tissue ingrowth within the inner wall of the native blood vessel and/or on the outer surface of the cover/tissue/material. For example, the cover can comprise the tissue/fabric of the anchoring region 610, which can be disposed on the outer surface of the frame 608(A) and may also be disposed on the inner surface of the frame 608(A). Any cover/material/tissue disposed on an inner surface of the frame 608(A) may be configured to either inhibit tissue ingrowth or merely not promote tissue ingrowth, if tissue growth within the frame 608(A) or the anchoring region 610 is not desired.

Referring back to FIG. 6A, in examples, the anchoring feature 608 is implemented as/with a docking station/structure 608(B)(also referred to as “a valve docking station 608(B)”) configured to receive/couple to a prosthetic valve, or other implantable structure. In an example, the prosthetic valve of the docking station 608(B) can provide a replacement for a native valve. Further, in examples, the prosthetic valve of the docking station 608(B) can provide functionality to implement a desired blood flow characteristic within the blood vessel. The blood flow characteristics of one or two prosthetic valves coupled to the device 600 can be tuned/designed/configured to collectively satisfy certain criteria, such as to control blood flow pressure through the prosthetic valves in a cooperative manner. In examples, a flow analysis can be implemented (e.g., simulated or actual) to determine the types of prosthetic valve(s) to implement (e.g., the characteristics of the prosthetic valves). In examples, two prosthetic valves at separate ends of the midsection 602 can assist in allowing blood to at least temporarily collect within the midsection 602 or to provide other desired results.

In examples, the docking station 608(B) can facilitate an implantable device such as a prosthetic valve to be more easily replaced over time, such as in the case where a first prosthetic valve wears out and needs to be replaced with a newer prosthetic valve, where a different prosthetic valve is needed due to different health issues of the patient, and so on. For instance, since patient tissue may not grow over the implantable device in the docking station 608(B), such implantable device can be removed more easily from the docking station 608(B) without disrupting the tissue.

The docking station 608(B) can include an outer frame/stent 608(B)(1) configured to secure the docking station 608(B) to the target anatomy (which can be similar to or the same as the frame 608(A)), and a seat/receiving portion 608(B)(2) configured to receive an implantable device 608(B)(3). Any of the features of the frame 608(A) may be included, as well as other features. The outer frame 608(B)(1) can be made of any at least partially rigid material, such as metal, plastic, etc. For example, the outer frame 608(B)(1) can comprise stainless steel or nitinol. Where nitinol or other shape-memory metal or material is implemented, the outer frame 608(B)(1) can be self-expanding. For instance, an expandable stent-type frame can be configured to expand radially from a compressed delivery configuration to the expanded state illustrated at 608(B). As shown, the outer frame 608(B)(1) and seat 608(B)(2) can be coupled together to form a channel through a central portion of the docking station 608(B)(where the implantable device 608(B)(3) can be located) and/or to prevent blood flow from flowing around the external walls of the outer frame. That is, the docking station 608(B) can seal to the target tissue to create a channel for blood to flow through the implantable device 608(B)(3), if present. In some instances, the outer frame 608(B)(1) is rolled back/bent (e.g., 180 degrees) to form the channel and/or seat 608(B)(2). In examples, the outside or inside of the docking station 608(B) is covered with a fabric, polymer cover, or other material, in like manner as discussed with reference to the frame 608(A).

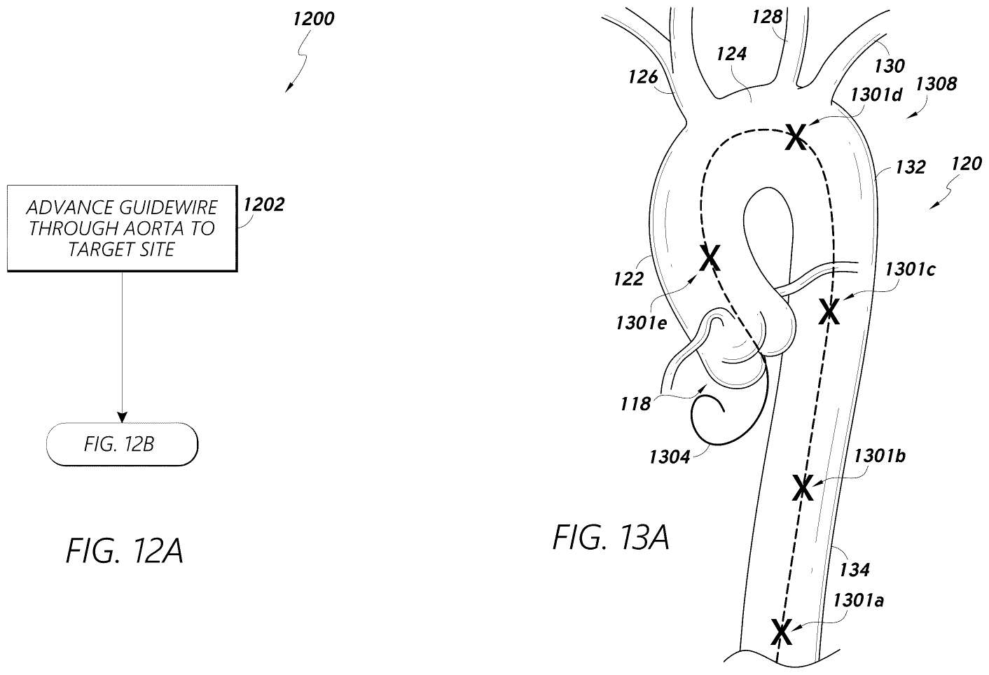

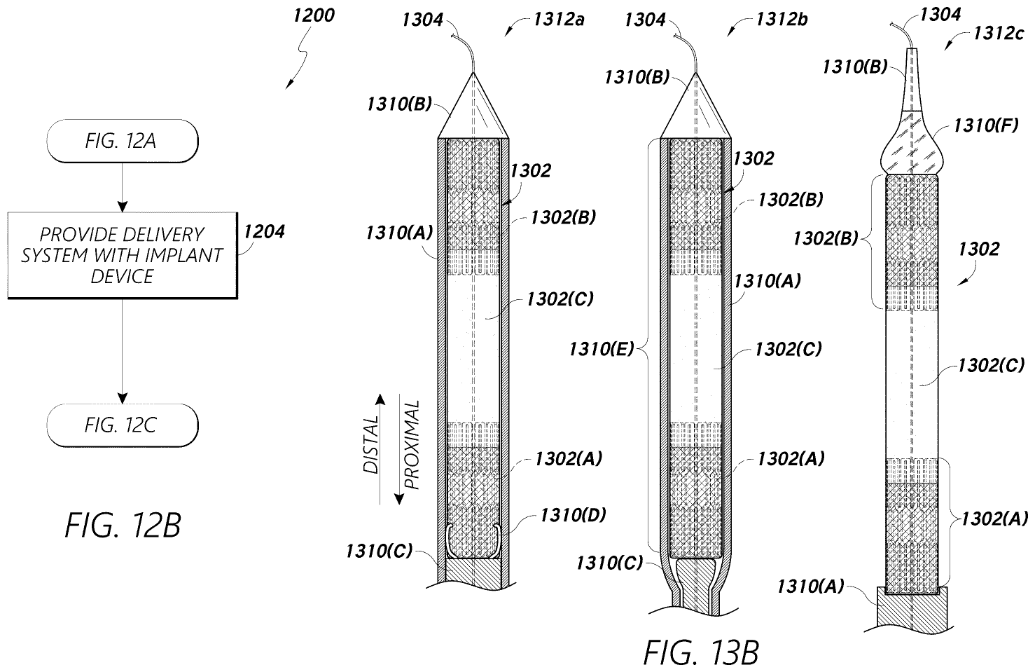

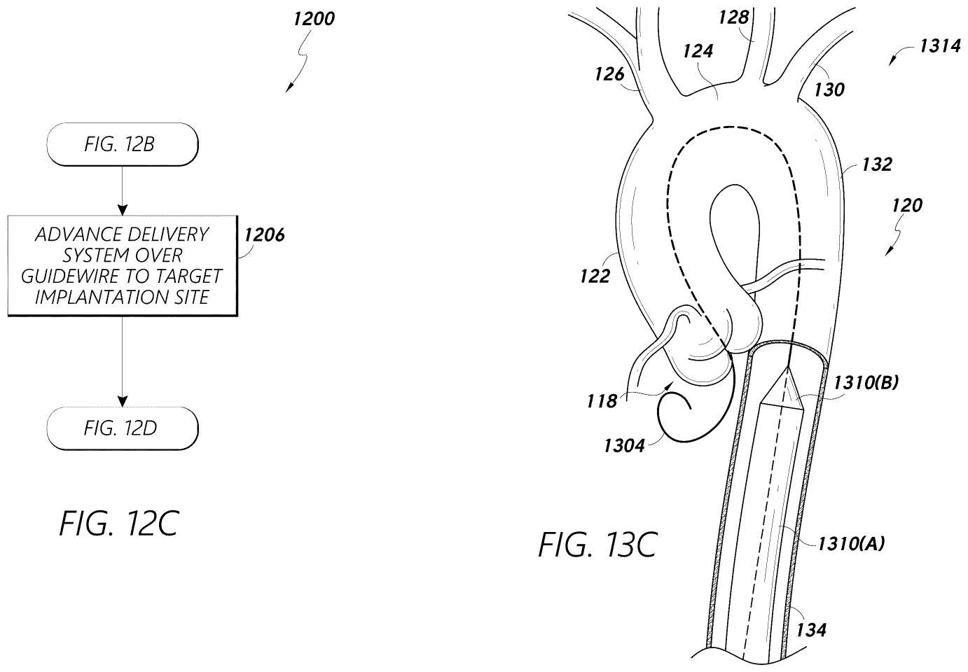

Further, as shown at FIG. 6A, in examples, the anchoring feature 608 is implemented as/with a prosthetic valve 608(C). The example prosthetic valve 608(C) can include a variety of forms, such as any of the prosthetic valves discussed herein, including the example prosthetic valves of FIGS. 7B, 7C, and 7D and/or the example prosthetic valve of FIG. 8. In examples, the prosthetic valve 608(C) can include substantially the same/similar dimensions as a native valve, since the prosthetic valve 608(C) is generally not implanted within a native valve. However, the prosthetic valve 608(C) can have other forms/dimensions. In examples, the prosthetic valve 608(C) can include an anchoring structure to anchor to patient tissue, such as that illustrated in FIG. 8, and/or the device 600 can include a cage/protective frame structure around the prosthetic valve 608(C) to prevent anatomy from disrupting the prosthetic valve 608(C).

Referring to the side view at FIG. 6D and the end view at FIG. 6E, if present, the form/wire form 612 (also referred to as “the form segment 612,” “the internal form 612,” or “tube form 612”) can include a frame-like structure comprising a plurality of struts/bars/wires configured in a tube-like shape. In some cases, the form 612 can be configured to prevent the midsection 602 from radially compressing/collapsing. In some instances, such as that shown in FIGS. 6D and 6E, the form 612 includes longitudinal/elongate struts that are oriented around a perimeter and cell struts forming one or more cells between the longitudinal struts. The cell struts can have any suitable or desirable shape (e.g., oval/ellipse, diamond/rhombus, hexagonal diamond/polygon, etc.). The cells can be arranged in any number of columns in the circumferential direction and/or rows in the axial, or lengthwise, direction. In examples, the form 612 can be at least somewhat convex in profile to assist in causing the midsection 602 to expand, instead of compressing (e.g., during systole). Although the form 612 is discussed in many examples as being positioned within the midsection 602, the form 612 can be positioned outside/external to the midsection 602.

The form 612 can be made of any at least partially rigid material, such as metal, plastic, etc. For example, the form 612 can comprise stainless steel or nitinol. Where nitinol or other shape-memory metal or material is implemented, the form 612 can be self-expanding. For instance, the form 612 as an expandable stent-type frame can be configured to expand radially from a compressed delivery configuration to the expanded state shown. In some implementations, the form 612 is formed from a sheet of metal, which is rolled into a cylinder to form the tubular/cylindrical form of the form 612. In examples, the form 612 can be implemented as a self-expandable, balloon/device expandable, or otherwise expandable frame. The form 612 can be formed using any suitable process, such as by stamping or machining the frame structure from a sheet or tube of metal.

When present, the outside or inside of the form 612 is covered with a fabric, polymer cover, or other material of the midsection 602. The midsection 602 material can be applied over or within the form 612 in any suitable or desirable manner. For example, the midsection 602 material can be applied using an electrical or mechanical spinning (e.g., rotary jet spinning, electrospinning, or similar) application process or other deposition process. In examples, one or more other layers of material can be implemented in addition to the midsection 602, which can help prevent the midsection 602 from radially compressing (e.g., through openings in the cells).

In some instances, the form 612 includes a relatively small number of struts/wires (in comparison to a threshold or typical stent). For example, the form 612 can include a few longitudinal struts and a few cell struts to hold the longitudinal struts in place, as shown in FIGS. 6D and 6E. In examples, the form 612 can include less struts than a frame 608(A), since the form 612 may not (in some instances) contact the anatomy in the patient or otherwise be subjected to external forces like the frame 608(A). To illustrate, the form 612 can include sufficient strut structure to prevent the midsection 602 from deflating or otherwise falling into the central axial channel of the device 600, which may be less strut structure than that needed to anchor a stent to a target anatomy.

In the examples shown, the midsection 602 extends over the form 612 to provide a sealed path for blood flow through the device 600. Here, the midsection 602 is attached/coupled to the anchoring regions 610; however, the midsection 602 can be attached/coupled at other locations and/or in other manners to seal the internal channel of the device 600. The midsection 602 and the anchoring regions 610 can be attached/coupled together in a variety of manners. In some cases, the midsection 602 extends completely over one or both of the anchoring regions 610. The midsection 602 can be disposed around or inside at least a portion of the anchoring region(s) 610 and/or the internal form 612.

In examples, at least a portion of the device 600 includes a concave form (or convex, in some cases) in profile, as shown in the example of FIGS. 6A-6E. Here, the internal form 612 (if present) and/or the midsection 602 (and/or the anchoring region(s) 610) is at least partially concave such that a medial portion of the form 612 and/or the midsection 602 has a smaller diameter than one or more ends/end portions of the form 612 and/or the midsection 602. This can minimize an amount of volume/space that the device 600 takes up within the blood vessel, such as the aorta, and/or maximize the amount of change in volume that the device 600 can provide. For instance, the medial section of the midsection 602 can be configured to expand from a concave form (in a non-pressurized state) to a straight or convex form (in a pressurized state). However, in some cases the form 612 and/or the midsection 602 does not include a concave form. Further, in some examples, the medial portion of the midsection 602 is more elastic than one or more end portions of the midsection 602.

FIGS. 7B, 7C, and 7D illustrate example prosthetic valves 604 that can be implemented as an anchoring feature 608(C) with the implant device 600, as described above and shown in FIG. 7A and elsewhere, in accordance with one or more examples. As with other anchoring features 608, the example prosthetic valves 604 can be implemented at either end of the device 600 (or at both ends). In some implementations, the device 600 and/or the prosthetic valve 604 can be mechanically expanded or radially self-expand from a compressed delivery state to the operational state under its own resiliency when released from a delivery system. In examples, the prosthetic valve 604 can be similar in one or more respects to the Edwards Lifesciences SAPIEN XT™, SAPIEN 3™, and/or SAPIEN 3 Ultra™ transcatheter prosthetic heart valves.

The prosthetic valve 604 can include valve leaflets/structure/feature(s) 702 supported inside a frame/frame structure/segment 704 (also referred to as “the valve frame 704”). The valve leaflets 702 can function by opening to permit flow in the presence of a pressure gradient in the direction of the prosthetic valve 604, such that the prosthetic valve 604 opens to permit flow and closes with each cardiac cycle to prevent flow in the opposite direction. The valve leaflets 702 can comprise any suitable or desirable material(s), such as biological tissue, polymer materials, etc. The prosthetic valve 604 can comprise any suitable or desirable number of leaflets, such as three, as shown in the illustrated examples. Although the prosthetic valve 604 is illustrated as including valve leaflets 702, other types of flow-control mechanisms can be utilized to achieve a desired direction and/or rate of flow through the device 600 in accordance with aspects of the present disclosure.

The valve frame 704 can include an annular structure having a plurality of vertically extending commissure attachment posts 706, which attach and/or help shape the leaflet structure 702 therein. Additional vertical posts or strut members 708, along with circumferentially extending strut members 710 can help form the rest of the valve frame 704. In some examples, the struts 708 and/or 710 can form axial rows of cells 712, which can be circumferentially staggered/offset, or the rows of cells 712 can be generally circumferentially aligned. The cells 712 can have any suitable or desirable shape (e.g., oval/ellipse, diamond/rhombus, hexagonal diamond/polygon, etc.). The open cell geometry of the valve frame 704 can facilitate coronary access, in some cases. With further reference to the example of FIG. 7D, the strut members 708/710 can form chevron/zig-zag shapes/cells in a base portion of the valve frame 704. The struts 708/710 can form edged crown portions 714 or apices at the inflow and/or outflow ends of the valve frame 704.

The valve frame 704 can be made of any at least partially rigid material, such as metal, plastic, etc. For example, the valve frame 704 can comprise stainless steel or nitinol. Where nitinol or other shape-memory metal or material is implemented, the valve frame 704 can be self-expanding. For instance, an expandable stent-type frame can be configured to expand radially from a compressed delivery configuration to the expanded state shown. In some implementations, an array of struts is formed from a sheet of metal, which is rolled into a cylinder to form the tubular/cylindrical form of the valve frame 704. The valve frame 704 can be formed using any suitable process, such as by stamping or machining the frame structure from a sheet or tube of metal. In some implementations, the valve frame 704 can be plastically expandable to its functional size by a balloon or another expansion device, in which case the frame 704 can be made of a plastically expandable material, such as stainless steel or a cobalt chromium alloy. Other suitable materials can also be used.

As described above, the valve frame 704 can be designed to be radially crimped or compressed to facilitate endovascular delivery to the target implant site. For example, the valved portion 606 can be positioned within a native blood vessel (e.g., aorta), where the valve frame 704 can be expanded to an operational state, for example, by an expansion balloon, such that the leaflet structure 702 or other flow-control mechanism of the valved portion 606 regulates blood flow through the native blood vessel or other anatomy.

In some implementations, the valve frame 704 is at least partially covered within and/or without by a sealing skirt 716, which can comprise any suitable or desirable material, such as textured polyethylene terephthalate (PET). The skirt 716 can be attached to an inner surface of the valve frame 704 to form a suitable/desirable attachment surface for the valve leaflets 702. The skirt 716 can be attached to the valve frame 704 in any suitable or desirable manner, such as through use of adhesive or other attachment means. In some examples, the skirt 716 is attached to an inner and/or outer surface of the valve frame 704 via one or more sutures 718, which can be wrapped around the various struts of the valve frame 704. The skirt 716 can provide a relatively more substantive attachment surface for portions of the leaflet structure 702 positioned closer to an inflow end 720 of the device 600.

In some examples, as shown in FIGS. 7B and 7C, the skirt 716 can be folded over the inflow end 720 of the frame 704 to cover over an outer diameter/surface of the frame 704. The portion of the skirt 716 on the outside of the frame 704 can facilitate attachment to the patient anatomy, such as through tissue ingrowth and/or frictional fit between the frame 704 and the anatomy (e.g., aorta or other blood vessel). In examples, the midsection 602 can be coupled/attached to the skirt 716 to seal the midsection 602 to the prosthetic valve 604 and provide a sealed channel for fluid/blood flow through the device 600. However, the midsection 602 can be attached to the prosthetic valve 604 in other manners.

In an example, the form 612 can extend from the outflow end 722 of the prosthetic valve 604, as shown in FIGS. 7B, 7C, and 7D. For example, the frame 704 of the prosthetic valve 604 can be coupled to or integral with the form 612, such as coupled via struts/elements 706, 708, 710, 714, etc. The midsection 602 (not shown in the right-side images of FIGS. 7B, 7C, and 7D) can extend over at least a portion of the frame 704 of the prosthetic valve 604 and attach to the frame 704, the skirt 716, and/or other portions of the prosthetic valve 604.

As noted above, although discussed in the context of the prosthetic valve 604, any of the example prosthetic valves of FIGS. 7B, 7C, and 7D can additionally, or alternatively, be implemented at either or both ends 610 of the device 600,

FIG. 8 illustrates another example prosthetic valve 604 that can be implemented with an implant device 600 in accordance with one or more examples. Here, the example prosthetic valve is discussed and illustrated in the context of the prosthetic valve 604 (a valved portion 606) of the device 600. However, the example prosthetic valve 604 can additionally, or alternatively, be implemented at one or both ends 610 of the device 600, as an anchoring feature 608. As similarly discussed above, in some implementations, the device 600 and/or the prosthetic valve 604 can be mechanically expanded or radially self-expand from a compressed delivery state to the operational state under its own resiliency when released from a delivery system. In examples, the prosthetic valve 604 can be similar in one or more respects to the Edwards Lifesciences SAPIEN XT™ SAPIEN 3™, SAPIEN 3 Ultra™, and/or Evoque transcatheter prosthetic heart valves.

As shown in FIG. 8, the prosthetic valve 604 can include one or more arms/elongate members 802 that are coupled to a frame 804 of the prosthetic valve 604 (which can be similar to or the same as the frame 704 discussed in reference to FIGS. 7B, 7C, and 7D). The one or more arms 802 can be distributed circumferentially around the frame 804 and/or attached to an inflow end of the prosthetic valve 604. The one or more arms 802 can initially project/extend from the frame 804 radially and then project/extend upwards/longitudinally (relative to FIG. 8) towards an outflow end of the prosthetic valve 604, as shown. At the end of each of the one or more arms 802, the respective arm 802 can include additional anchoring features 806 configured to anchor/secure the prosthetic valve 604 to patient anatomy, such as to the tissue/wall of a native blood vessel. The additional anchoring feature(s) 806 can include a barb(s), patch(es), pin(s), coil(s), screw(s), tab(s), hook(s), wire(s), spike(s), or other tissue anchor means configured to embed in and/or hold to the anatomy.

In examples, the one or more arms 802 and/or the one or more additional anchoring features 806 can assist in holding/maintaining the prosthetic valve 604 in a relatively secure manner, such as to prevent the prosthetic valve 604 from moving around within the patient anatomy. In examples, the one or more arms 802 and/or the one or more anchoring features 806 can be configured to mechanically expand or radially self-expand from a compressed delivery state to the operational state shown in FIG. 8. The one or more arms 802 and/or the one or more additional anchoring features 806 can provide anchoring functionality to anchor the prosthetic valve 604 to target tissue.

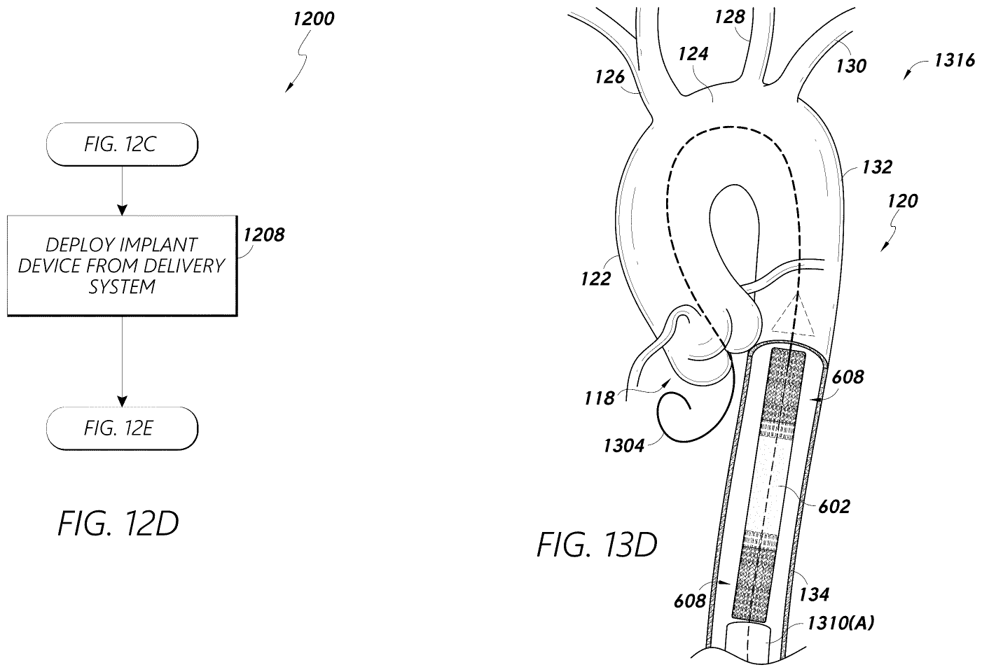

FIG. 9 illustrates the device 600 disposed/implanted within example anatomy of a patient, namely the descending thoracic aorta 132, in a cross-sectional view of the aorta 120, in accordance with one or more examples. In various examples, the device 600 can be implanted along any suitable aortic segment or any other blood vessel segment (that does not include side branches that may be otherwise occluded by the device 600), or in other patient anatomy. In an example, each end/anchoring region 610 of the device 600 is anchored to the inner wall of the blood vessel (e.g., aorta 120). The midsection 602 of the device 600 may not be anchored to the wall of the blood vessel, and may be “floating” with respect to the blood vessel wall. The device 600 may be implanted in any orientation (i.e., with regard to cither end of the device 600 and the orientation of the blood vessel and blood flow from the heart 100), or may be implanted in a specific orientation, depending on whether particular anchoring features 608 are present at one or both ends of the device 600. For instance, in the example shown at FIG. 9, both anchoring regions 610 of the device 600 include an anchoring feature 608(A) comprising a stent-like device. In the example, the device 600 can be implanted in either orientation, with either end/anchoring region 610 upstream or downstream relative to the flow of blood from the heart 100. In a case where one or both ends include an anchoring feature 608(B) or an anchoring feature 608(C), which can include a prosthetic valve, the device 600 can be positioned/deployed so that one end is closer to the aortic arch 124 or the heart 100 than the other, to provide desired functionality and blood flow through the valve(s) and the device 600 during the cycles of the heart 100. In an example, one end of the device 600 having a valve 608(C) may be positioned upstream within the blood vessel with the second end positioned downstream relative to the flow of blood through the blood vessel to provide desired valve functionality and blood flow through the valve(s) and the device 600.

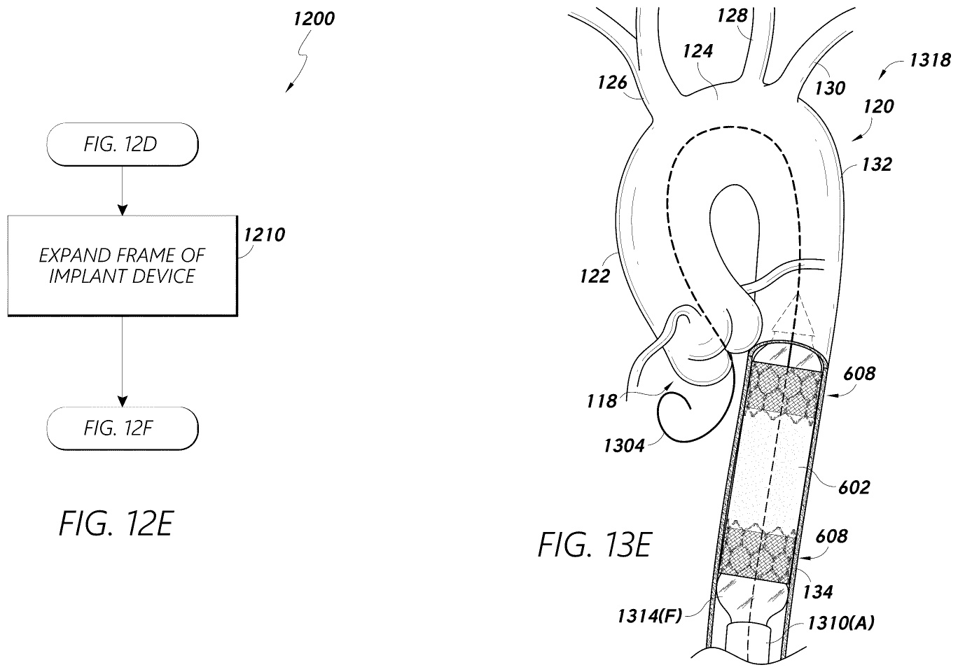

FIGS. 10A and 10B illustrate operation of the device 600 as implanted in the descending thoracic aorta 132 during various phases of the cardiac cycle and in accordance with one or more examples. FIG. 10A depicts expansion of the device 600 during at least a portion of systole, while FIG. 10B depicts contraction of the device 600 during at least a portion of diastole.

As shown in FIG. 10A, during systole, the aortic valve 118 is open to allow blood flow through the aorta 120 and through the device 600 when the ventricles of the heart 100 contract. As blood is forced into the device 600, the blood/luminal pressure within the midsection 602 increases and the midsection 602 expands, increasing the volume of the device 600 and thereby storing blood and energy within the midsection 602 in the form of elastic stretch. In examples, the form 612 (if present) can assist in preventing the midsection 602 from compressing inward during systole or at other times.

In contrast, as shown in FIG. 10B, during diastole, the aortic valve 118 is closed and the mitral valve 116 is open to allow blood to flow into the left ventricle 104 from the left atrium 102, as the ventricles relax. Here, the blood/luminal pressure within the midsection 602 decreases, resulting in the midsection 602 radially compressing and releasing the blood from within the device 600 into circulation within the aorta 120. That is, the release of stored energy in the midsection 602 causes the midsection 602 to contract/recoil back towards the original state thereof due to decreasing blood pressure within the aorta 120 and within the device 600. The action of the midsection 602 contracting during diastole can generate pressure that pushes blood to the organs of the body, including pushing blood through the cardiac arteries to the heart muscles.

As disclosed herein, the device 600 can include an axial internal central fluid channel within the midsection 602 that is configured to radially expand and retract/recoil based on changing pressure conditions within the channel (e.g., changing luminal pressures). As the midsection 602 radially expands and contracts, the volume of the channel increases and decreases in a manner to provide compliance for the blood vessel in which it is implanted, such as the aorta 120 in this example.

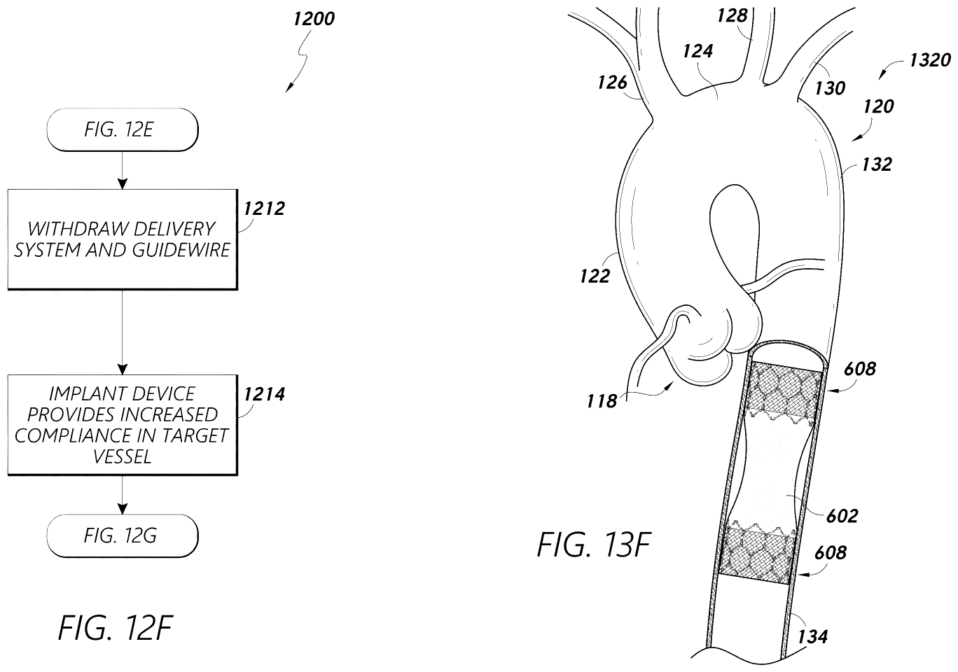

FIG. 11 illustrates the device 600 disposed/implanted within an aged and/or stiffened aorta 120, as shown in FIG. 1C, in accordance with one or more examples. The vasculature of the aorta 120 (or other blood vessel) has become less elastic, wherein the compliance of at least a portion of the vessel is diminished relative to a healthy aorta 120 as shown in FIG. 1B. As such conditions develop, arterial blood pressure (e.g., left-ventricular afterload) can become more pulsatile, which can have deleterious effects, such as the thickening of the left ventricle (LV) muscle, and insufficient perfusion of the heart 100. Insufficient perfusion of the heart muscle can lead to and/or be associated with heart failure. Such conditions can result in reduced cardiac output and/or elevated intra-cardiac pressures at rest or during stress. The device 600 can be implanted along a suitable aortic segment (or other blood vessel segment) where it is desired to improve the elasticity and compliance of the blood vessel. For instance, a stiff or at risk section 1100 of the blood vessel may be selected as the target site. In an example, each end/anchoring region 610 of the device 600 is anchored to the inner wall of the blood vessel (e.g., aorta 120) at the target site. The device 600 may be disposed such that the anchoring regions 610 are positioned on either end of (e.g., bordering or flanking) the stiff or at risk section 1100 of the blood vessel. The midsection 602 of the device 600 may not be anchored to the wall of the blood vessel, and may be “floating” with respect to the blood vessel wall.

As part of the deployment of the device 600 within the patient anatomy, the wall of the aorta 120 (or other blood vessel or anatomy) may be intentionally ruptured/torn and the midsection 602 of the device 600 disposed at the site of the tear 1110. Positioning the device 600 at the tear 1110 can give room for the midsection 602 to expand to a desired volume during increased blood pressure (e.g., during systole). For example, without the rupture/tear 1110, the target site may not be sufficiently compliant (due to the stiffness of the stiff or at risk section 1100 of the blood vessel), which can limit the expansion of the midsection 602, rendering it less effective at improving the compliance of the blood vessel. Thus, the midsection 602 is disposed at the location of the rupture 1110. The anchoring regions 610 border the rupture 1110, sealing the aorta 120 on either end of the rupture 1110. The fabric at the midsection 602 provides sufficient sealing to prevent leakage therethrough, thereby ensuring continuous aortic blood flow through the device 600 at the location of the rupture 1110 and through the aorta 120. The stretchable fabric of the midsection 602 allows the midsection 602 to radially expand into the torn section 1110 under elevated systolic blood pressure to a desired volume. The diameter of the midsection 602 increases, which presents a greater cross-sectional area of the blood vessel at the midsection 602. The expansion at the midsection 602 thereby adds compliance to the blood vessel to reduce pulsatile LV afterload in hypertensive HF patients. The midsection 602 reverts to a contracted state when pressure is reduced during diastole. The diameter of the device 600 (and particularly the midsection 602 of the device 600) is similar to that of the natural blood vessel, which substantially maintains the cross-sectional area of the blood vessel. In this manner, the previously relatively stiff (i.e., non-compliant) aortic section 1100, now torn, is replaced by the device 600, which is a pliant component that can radially expand and contract in response to the changes in blood pressure. Further, since the fabric of the midsection 602 is configured to discourage tissue ingrowth (e.g., is thromboresistant) to mitigate any tissue ingrowth between the midsection 602 and the aortic wall, the midsection 602 can remain pliant and resist becoming stiff and non-compliant as well. For example, preventing the buildup of tissue on the flexible material of the midsection 602 can prevent a reduction in the flexibility of the material that can reduce the compliance of the midsection 602.

The elastic material of the midsection 602 can be longer than the fabric at the proximal and distal anchoring regions 610, and is configured to be stretchable and compliant. The material of the proximal and distal anchoring regions 610, on the other hand, can be relatively stretched, but may not be selected for substantial stretching capability. The flexible material of the midsection 602 may be under some tension in the relaxed state, or it may be without tension, depending on the material selected and the construction, as desired for closely imitating the expansion and contraction of a healthy blood vessel such as the aorta 120.

Although the device 600 is illustrated with an anchoring feature 608(A) comprising a stent-like device at both ends in the examples of FIGS. 9, 10A, 10B, and 11 (and elsewhere), the device 600 can alternately include a prosthetic valve at one or both ends of the device 600, as discussed herein.

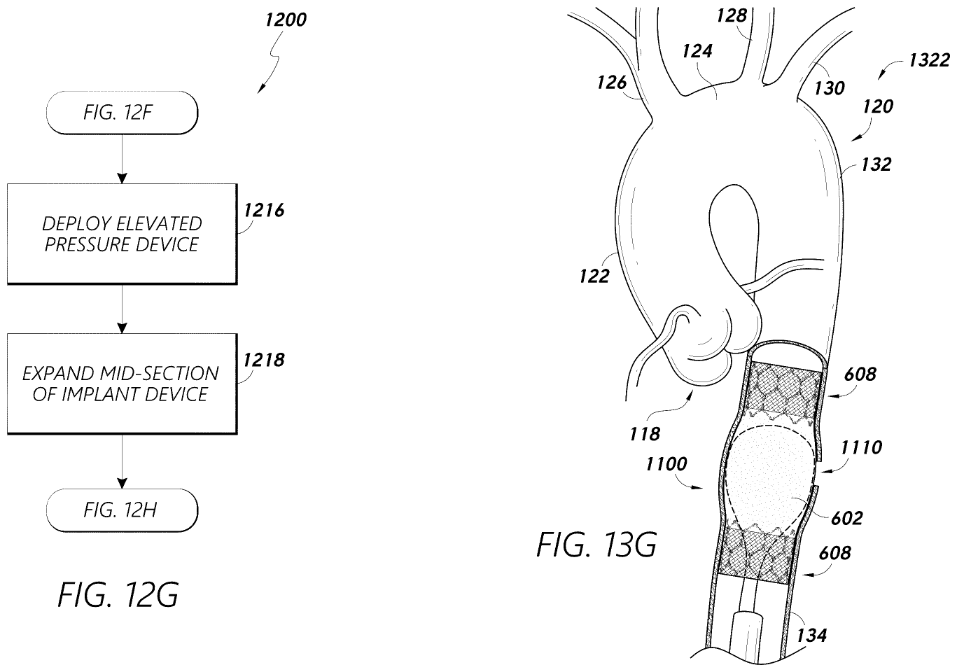

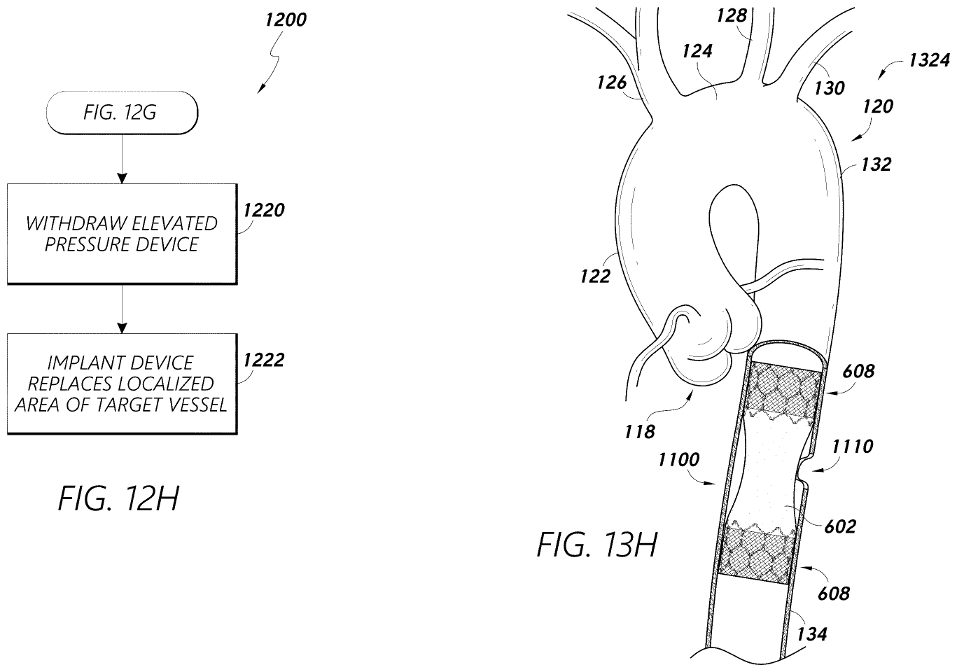

FIGS. 12A, 12B, 12C, 12D, 12E, 12F, 12G, and 12H illustrate a flow diagram for a process 1200 for implanting an implant device (such as the implant device 600) in accordance with one or more examples. FIGS. 13A, 13B, 13C, 13D, 13E, 13F, 13G, and 13H provide images of an example implant device 600 and certain anatomy corresponding to operations of the process 1200 of FIGS. 12A, 12B, 12C, 12D, 12E, 12F, 12G, and 12H according to one or more examples. The process 1200 utilizes a transcatheter procedure for implantation/deployment of compliance-enhancement implant devices in accordance with aspects of the present disclosure. However, implant devices disclosed herein can be implanted using other types of minimally-invasive and/or surgical procedures. The implant device 600 illustrated in the figures can be representative of any of the examples of implant devices discussed herein, or any other implant device.

At block 1202, the process 1200 includes advancing a guidewire 1304 through at least a portion of the aorta 120 (and/or the aortic valve 118) of the patient to reach a target implantation site. For example, image 1308 shows an example implantation site 1301a in the abdominal aorta 134, an example implantation site 1301b between the descending thoracic aorta 132 and the abdominal aorta 134, an example implantation site 1301c in the descending thoracic aorta 132, an example implantation site 1301d in the aortic arch 124, and an example implantation site 1301e in the ascending aorta 122. The guidewire 1304 can also be advanced through the aortic valve 118 and/or into the left ventricle 104 to an implantation site if desired. Access to an implantation site can be made through any suitable vessel puncture providing access to the arterial system. For example, access can be made via the femoral artery or other (e.g., arterial) blood vessel.

Although the process 1200 and certain other examples are described herein in the context of implantation within the aorta 120, compliance-enhancement devices of the present disclosure can be implanted in other arterial or venous blood vessels, such as the inferior vena cava. Further, although the process 1200 and accompanying illustrations are presented with respect to the implantation of a single compliance-enhancement implant device 600, the process 1200 can involve implanting multiple compliance-enhancement implant devices in various positions within the aorta 120 and/or other blood vessel(s).

In FIG. 12B, at block 1204, the process 1200 includes providing a delivery system 1302 having the implant device (e.g., implant device 600) disposed in a distal portion thereof. Images 1312a, 1312b, and 1312c of FIG. 13B show a cut-away view of implementations of the delivery system 1302 in accordance with one or more examples. The delivery system 1302 can comprise one or more catheters, sheaths, balloons, and/or other devices used to advance and/or implant the implant device 600, which can be disposed at least partially within the delivery system 1302 during portions of the process 1200. The implant device 600 can be positioned within the delivery system 1302 with a first end thereof (e.g., a first anchoring region 610) disposed proximally and a second end (e.g., a second anchoring region 610) disposed distally with respect to the illustrated orientation of the delivery system 1302.

In some examples, the delivery system 1302 comprises an outer catheter/shaft/sheath 1310(A), which can be used to transport the compliance-enhancement implant device 600 to the target implantation site. That is, the compliance-enhancement implant device 600 can be advanced to the target implantation site at least partially within a lumen of the outer shaft 1310(A), such that the compliance-enhancement implant device 600 is held and/or secured at least partially within a distal portion of the outer shaft 1310(A) in a radially compressed configuration.

In some examples, the delivery system 1302 comprises a tapered nosecone feature 1310(B), which can facilitate advancement of the distal end of the delivery system 1302 through the tortuous anatomy of the patient and/or an outer delivery sheath or other conduit/path. The nosecone 1310(B) can be a separate component from the outer shaft 1310(A) or can be integrated with the outer shaft 1310(A). In some examples, the nosecone 1310(B) is adjacent to and/or integrated with a distal end of the outer shaft 1310(A). In some examples, the nosecone 1310(B) is distally tapered into a generally conical shape and can comprise and/or be formed of multiple flap-type forms that can be urged/spread apart when the compliance-enhancement implant device 600 and/or any portions thereof, interior shafts, or devices, are advanced distally therethrough.

The delivery system 1302 can further be configured to have the guidewire 1304 disposed at least partially within the delivery system 1302 and/or coupled thereto in a manner to allow the delivery system 1302 to follow a path defined by the guidewire 1304. In some implementations, the guidewire 1304 can pass through an interior of the implant device 600 and/or through a lumen of a pusher device or tube of the delivery system 1302.

In the examples of images 1312a and 1312b, the implant device 600 is configured to be compressed/collapsed and disposed within the shaft/sheath 1310(A) in the radially compressed/collapsed configuration. The device 600 in the compressed form is shown at 1302(A), 1302(B), and 1302(C), where 1302(A) and 1302(B) represent the compressed anchoring regions 610 and 1302(C) represents the compressed midsection 602. Any anchoring features 608(A), 608(B), or 608(C), and/or internal form 612 are crimped or compressed to assume a reduced radial profile within 1302(A), 1302(B), and/or 1302(C). In the compressed delivery configuration, the device 600 can be somewhat more elongated compared to a fully expanded configuration thereof due to at least some of frame structure of the device 600 being deflected into more longitudinally-oriented configurations. In the example of image 1312c the implant device 600, represented as 1302(A), 1302(B), and 1302(C), can be compressed/collapsed and/or held between a balloon 1310(F) and the sheath 1310(A).

In some cases, the delivery system 1302 can comprise a pusher shaft 1310(C), which can be slidingly disposed within the outer sheath 1310(A) proximal and/or adjacent to the implant device 600. The pusher 1310(C) can be configured to be used to push/advance the implant device 600 relative to the outer shaft/sheath 1310(A) to deploy the device 600 from the sheath 1310(A). For example, the pusher 1302(C) can be distally advanced relative to the outer sheath 1310(A) to cause distal advancement of the implant device 600 through a distal opening in the outer sheath/shaft 1310(A). Alternatively or additionally, the implant device 600 can be deployed from the outer sheath 1310(A) at least in part by proximally pulling the outer sheath 1310(A) relative to the pusher 1310(C).

The image 1312a illustrates an example implementation of the delivery system 1302 that includes one or more coupling/attachment elements 1310(D) to releasably attach to the implant device 600, represented as 1302(A), 1302(B), and 1302(C). For example, the coupling elements 1310(D) can comprise one or more feet or arms that project distally and/or radially from the pusher 1310(C). In some cases, the pusher 1310(C) is releasably attached to the implant device 600. After the device 600 has been deployed from the sheath 1310(A), positioned in the desired implantation site/position, and/or expanded, the pusher 1310(C)/coupling element(s) 1310(D) (or other component of the delivery system 1302) can be disengaged from the implant device 600 to release the device 600 and allow for removal/withdrawal of the delivery system 1302. Although coupling elements 1310(D) are illustrated, these elements can be eliminated.

The image 1312b shows an example implementation of the delivery system 1302 that includes a distal capsule portion 1310(E), wherein the implant device 600, represented as 1302(A), 1302(B), and 1302(C), is disposed in the compressed configuration within the capsule portion 1310(E). Deployment of the implant device 600 can be implemented at least in part by proximally pulling the outer sheath 1310(A) such that the implant device 600 is pressed proximally against the pusher 1310(C) to keep the implant device 600 in place while unsheathing is taking place. In some implementations, the capsule portion 1310(E) can have a diameter that is greater than that of the outer sheath 1310(A) in the area proximal to the capsule 1310(E) (e.g., a more proximal part of the implant device 600).

The image 1312c shows an example delivery system 1302 that includes the balloon 1310(F), which can be positioned at least partially within the implant device 600 for delivery of the implant device 600. Here, the implant device 600, represented as 1302(A), 1302(B), and 1302(C), is configured to remain in a compressed state with minimal or no constraints (e.g., with the outer sheath 1310(A) covering just a portion or none of the implant device 600), wherein the implant device 600 is configured to be expanded by the balloon 1310(F) at a target site.

Although features are shown within respect to particular delivery system implementations, any of the delivery systems 1302 of images 1312a-c can include any of the features, even if such feature is not illustrated for a specific implementation.

In FIG. 12C, at block 1206, the process 1200 includes advancing the delivery system 1302 over the guidewire 1304 until the target implantation site is reached to thereby position the implant device 600 for deployment in the target anatomy, as shown in image 1314 of FIG. 13C. For instance, the delivery system 1302 can be advanced to position the device 600 to a stiff or at risk portion of the aorta 120.

In FIG. 12D, at block 1208, the process 1200 includes deploying the implant device 600 from the delivery system 1302. In examples, to deploy the implant device 600, the outer sheath 1310(A) is proximally pulled and/or the pusher 1310(C) is distally pushed to thereby draw the sheath 1310(A) past the distal end of the implant device 600, at least partially exposing/deploying the implant device 600, as shown in image 1316. For instance, initially the sheath 1310(A) can be withdrawn to position the implant device 600 at the target site, while maintaining position around and holding an anchoring feature 608 at the distal end if present. The sheath 1310(A) can be further withdrawn to position/anchor the distal anchoring feature 608 at the target location for anchoring the implant device 600. In some cases, the sheath 1310(A) can be further withdrawn to position an additional anchoring feature 608, if present on the proximal end of the implant device 600. The implant device 600 can comprise one or more radiopaque markers that can be referenced to determine/confirm the position of the implant device 600 at various stage(s) of the process 1200 using a suitable imaging modality. Although discussed in the context of implanting a device within the descending aorta 134, the implant device 600 can be implanted in other areas of the aorta 120 or in other anatomy.

The implant device 600, including the anchoring regions 610, the midsection 602, any anchoring features 608, and/or an internal form 612, and/or other elements of the implant device 600 can be implemented in a variety of manners. In examples, any of these elements can be self-expandable, such that portion of the implant device 600 is expanded/fully deployed (e.g., anchored/secured to target tissue) upon release from the outer sheath 1310(A). For instance, expansion of a portion of the implant device 600 can be achieved via shape memory features. To illustrate, one or more portions of the implant device 600 can comprise nitinol or other shape-memory metal configured to self-expand when released from the delivery sheath/capsule.

Further, in examples, the anchoring regions 610, the midsection 602, any anchoring features 608, and/or an internal form 612, and/or other elements of the implant device 600 can be expanded by a device/physician. In such cases, in FIG. 12E, at block 1210 and illustrated in FIG. 13E, the process 1200 includes expanding an element(s) of the implant device 600 to thereby secure the implant device 600 in place in the deployed/expanded configuration.

In examples, as shown in image 1318, the delivery system 1302 includes the balloon 1314(F) configured to expand one or more elements/portions of the implant device 600. The implant device 600 can be situated on the balloon 1314(F) such that when the balloon 1314(F) is inflated (to a minimal inflation pressure to expand the components of the device 600), one or more portions of the implant device 600 expand with the balloon 1314(F). The balloon 1314(F) can serve to expand frame struts/portions by pushing outwardly against the frame struts/portions. For instance, when the balloon 1314(F) is inflated within an anchoring feature 608, the anchoring feature 608 is expanded to a desired circumference to contact the inner walls of the aorta 120 (or other blood vessel). In examples, the balloon 1314(F) is positioned within the internal form 612, when such internal form 612 is implemented. After the balloon 1314(F) has expanded the implant device 600, the balloon 1314(F) can be deflated and removed.

Further, in examples, the implant device 600 or any elements/portions thereof can be expanded/dilated using pull wire(s) that are configured to be pulled or pushed to cause expansion of element(s). For example, a pull wire(s) can be coupled to the distal portion of an element(s) of the implant device 600 such that pulling the wire(s) proximally causes the ends of the element(s) to be brought together, thereby dilating/expanding the element(s).

In some cases, the expansion operation(s) associated with block 1210 can involve dilating the native blood vessel (before or after expanding the implant device 600) by expanding the implant device 600 or portion thereof to a diameter, at least with respect to a lengthwise portion thereof, that is greater than the diameter of the native blood vessel, wherein such dilation of the blood vessel can serve to form/introduce a space for radial expansion of one or more portions of the implant device 600.

In FIG. 12F, at block 1212, the process 1200 includes withdrawing the delivery system 1302 and/or guidewire 1304, leaving the implant device 600 implanted, as shown in image 1320 of FIG. 13F. With the implant device 600 implanted, the implant device 600 may be capable of providing increased compliance to thereby improve arterial blood flow and/or prevent elevated blood pressure, as shown at block 1214. Other benefits can also be achieved, as described herein. However, if the compliance of the blood vessel is diminished, the stiffness of the blood vessel may create limits on the ability of the midsection 602 to expand during higher pressure states. Accordingly, it can be desirable to form a rupture or tear in the blood vessel (or other anatomy) to allow the midsection 602 room to expand.

During a desired or predetermined waiting period, which can be used to allow for tissue ingrowth to occur over the outer surface of the proximal and distal anchoring regions 610 of the implant device 600, the fabric at the anchoring regions 610 is bonded to the interior tissue of the blood vessel wall, anchoring the device 600 at the target region 1100. The bonding may be further enhanced in the case where one or more anchoring features 608 of the implant device 600 are engaged with the tissue of the interior of the blood vessel wall. Meanwhile, the midsection 602 remains unattached to the blood vessel wall (i.e., “floating”) due to its thromboresistant properties. Referring to block 1216 of FIG. 12G and illustrated at FIG. 13G, after such waiting period, the process 1200 includes deploying another balloon 1314(F) (or other device) to the site of the implant device 600 and into the interior of the midsection 602. At block 1218, the balloon 1314(F) is inflated to an elevated pressure, greater than the pressure previously used at block 1210, expanding the circumference of the balloon 1314(F), which expands the midsection 602 of the implant device 600 and the interior of the blood vessel. The expansion of the midsection 602 is sufficient to cause a rupture 1110 and/or a tear of the blood vessel (or other anatomy) tissue surrounding the midsection 602. The rupture 1110 is precisely and carefully confined to the area of the blood vessel surrounding the midsection 602 of the implant device 600 by regulating the pressure of the balloon 1314(F). The anchoring regions 610 that border the rupture 1110 seal the aorta 120 on either end of the rupture 1110.

In FIG. 12H, at block 1220, the process 1200 includes deflating the balloon 1314(F) and withdrawing the balloon 1314(F) and delivery system 1302 and/or guidewire 1304, leaving the implant device 600 implanted, as shown in image 1324 of FIG. 13H. The rupture 1110 remains in the sidewall of the aorta 120 (or other blood vessel) to allow the midsection 602 room to expand as desired. At block 1222, the midsection 602 replaces the torn region 1110 of the aorta 120, providing a more compliant component in its place. As discussed above, the flexible material at the midsection 602 is sufficiently impervious to blood to prevent leakage through the material. Blood flows through the implant device 600 and on through the aorta 120 (or other blood vessel), bypassing the torn area 1110 of the aorta 120. In this manner, the previously relatively stiff (i.e., non-compliant) aortic section 1100, now torn, is replaced by a compliant component (the midsection 602) that can radially expand and contract in response to blood pressure changes, and thereby increase blood flow including during the diastolic phase. This can have the result of reducing pulsatile LV afterload in hypertensive patients.

The above method(s) can be performed on a living animal or on a simulation, such as on a cadaver, cadaver heart, anthropomorphic ghost, simulator (e.g., with body parts, heart, tissue, etc. being simulated). Any of the various systems, devices, apparatuses, etc. in this disclosure can be sterilized (e.g., with heat, radiation, ethylene oxide, hydrogen peroxide, etc.) to ensure they are safe for use with patients, and/or the methods herein can comprise sterilization of the associated system, device, apparatus, etc. (e.g., with heat, radiation, ethylene oxide, hydrogen peroxide, etc.).

### Additional Description of Examples

Provided below is a list of examples, each of which may include aspects of any of the other examples disclosed herein. Furthermore, aspects of any example described above may be implemented in any of the numbered examples provided below.

Example 1: An implant device comprising: a pliant tube including a first end and a second end, including: a first anchoring region disposed at the first end; a second anchoring region disposed at the second end, the first anchoring region and the second anchoring region configured to anchor the implant device within an anatomical structure and to encourage tissue ingrowth at an outer surface of the first anchoring region and an outer surface of the second anchoring region; and a flexible midsection disposed between the first anchoring region and the second anchoring region.

Example 2: The implant device of any example herein, in particular example 1, wherein the implant device is configured to extend within a segment of a blood vessel.

Example 3: The implant device of any example herein, in particular example 1 or example 2, further comprising: a first anchoring structure associated with the first anchoring region, configured to anchor the first anchoring region to an inner surface of the anatomical structure.

Example 4: The implant device of any example herein, in particular example 3, further comprising: a second anchoring structure associated with the second anchoring region, configured to anchor the second anchoring region to the inner surface of the anatomical structure.

Example 5: The implant device of any example herein, in particular example 3, wherein the first anchoring structure comprises an expandable stent.

Example 6: The implant device of any example herein, in particular example 3, wherein the first anchoring structure comprises a prosthetic valve or a docking device for a prosthetic valve.

Example 7: The implant device of any example herein, in particular example 3, further comprising a prosthetic valve associated with the second anchoring region, configured to anchor the second anchoring region to an inner surface of the anatomical structure.

Example 8: The implant device of any example herein, in particular example 1, wherein at least one of the first and second anchoring regions is comprised of a natural or synthetic fabric that is coated with a compound that encourages tissue ingrowth.

Example 9: The implant device of any example herein, in particular example 8, wherein an inner surface of the first and second anchoring regions is free from the compound that encourages tissue ingrowth.

Example 10: The implant device of any example herein, in particular example 8, wherein the natural or synthetic fabric is disposed over an expandable stent.

Example 11: The implant device of any example herein, in particular example 1, wherein at least one of the first and second anchoring regions is comprised of a natural or synthetic fabric that is fabricated from a compound that encourages tissue ingrowth.

Example 12: The implant device of any example herein, in particular example 11, wherein the natural or synthetic fabric is disposed over an expandable stent.

Example 13: The implant device of any example herein, in particular example 1, wherein the midsection is configured to radially expand in response to an increase in pressure within the anatomical structure and to contract elastically in response to a decrease in pressure within the anatomical structure.

Example 14: The implant device of any example herein, in particular example 1, wherein the midsection is configured to radially expand during systole and to contract elastically during diastole.

Example 15: The implant device of any example herein, in particular example 1, wherein the midsection is fabricated from a compound that inhibits tissue ingrowth.

Example 16: The implant device of any example herein, in particular example 1, wherein the midsection is comprised of a thromboresistant material or is coated with a thromboresistant material.

Example 17: An implant device comprising: a tubular graft having a proximal end and a distal end; a first anchoring region disposed at the proximal end, configured to encourage tissue ingrowth at an outer surface of the first anchoring region; a second anchoring region disposed at the distal end, configured to encourage tissue ingrowth at an outer surface of the second anchoring region; and a flexible midsection disposed between the proximal end and the distal end, and configured to inhibit tissue ingrowth at the flexible midsection.

Example 18: The implant device of any example herein, in particular example 17, wherein the first anchoring region and the second anchoring region are configured to anchor the implant device within a segment of a blood vessel.

Example 19: The implant device of any example herein, in particular examples 17 or 18, further comprising a first anchoring structure associated with the first anchoring region, configured to anchor the first anchoring region to an inner surface of the blood vessel.

Example 20: The implant device of any example herein, in particular example 19, further comprising a second anchoring structure associated with the second anchoring region, configured to anchor the second anchoring region to the inner surface of the blood vessel.

Example 21: The implant device of any example herein, in particular example 17, wherein the first anchoring region includes a first expandable stent configured to anchor the implant device to an inner surface of the blood vessel.

Example 22: The implant device of any example herein, in particular example 21, wherein the first expandable stent includes a first natural or synthetic tissue that is coated at an outer surface of the first natural or synthetic tissue with a compound that encourages tissue ingrowth.

Example 23: The implant device of any example herein, in particular example 21, wherein the first expandable stent includes a first natural or synthetic tissue that is fabricated from a compound that encourages tissue ingrowth.

Example 24: The implant device of any example herein, in particular examples 22 or 23, wherein a material of the midsection is different from the first natural or synthetic tissue.

Example 25: The implant device of any example herein, in particular example 21, wherein the second anchoring region includes a second expandable stent configured to anchor the implant device to an inner surface of the blood vessel.

Example 26: The implant device of any example herein, in particular example 25, wherein the second expandable stent includes a second natural or synthetic tissue that is coated at an outer surface of the second natural or synthetic tissue with a compound that encourages tissue ingrowth.

Example 27: The implant device of any example herein, in particular example 25, wherein the second expandable stent includes a second natural or synthetic tissue that is fabricated from a compound that encourages tissue ingrowth.

Example 28: The implant device of any example herein, in particular examples 26 or 27, wherein a material of the midsection is different from the second natural or synthetic tissue.

Example 29: The implant device of any example herein, in particular example 17, wherein the first anchoring region and the second anchoring region are configured to seal to an inner wall of a blood vessel to prevent fluid leakage from between the first anchoring region and the second anchoring region and the inner wall of the blood vessel.

Example 30: The implant device of any example herein, in particular example 17, wherein the midsection is comprised of a thromboresistant material or is coated with a thromboresistant material.

Example 31: An implant device comprising: a pliant tubular device configured to be implanted within an anatomical structure, having a first anchoring region at a proximal end, a second anchoring region at a distal end, and a flexible midsection disposed therebetween, the first and second anchoring regions being configured to encourage tissue ingrowth at an outer surface of the first and second anchoring regions and the flexible midsection configured to inhibit tissue ingrowth.

Example 32: The implant device of any example herein, in particular example 31, further comprising an internal form having a frame structure disposed within at least the flexible midsection and configured to prevent the flexible midsection from radially compressing.

Example 33: The implant device of any example herein, in particular examples 31 or 32, further comprising a first anchoring structure disposed at the first anchoring region, configured to anchor the first anchoring region to an inner surface of the anatomical structure.

Example 34: The implant device of any example herein, in particular example 33, further comprising a second anchoring structure disposed at the second anchoring region, configured to anchor the second anchoring region to the inner surface of the anatomical structure.

Example 35: The implant device of any example herein, in particular example 34, wherein at least one of the first and second anchoring structures comprises an expandable stent, a prosthetic valve, or a docking station for a prosthetic valve.

Example 36: The implant device of any example herein, in particular example 31, wherein at least one of the first and second anchoring regions is comprised of a natural or synthetic fabric that is fabricated using a compound that encourages tissue ingrowth or is coated with a compound that encourages tissue ingrowth.

Example 37: The implant device of any example herein, in particular example 31, wherein the pliant tubular device is configured to be implanted within a segment of a blood vessel.

Example 38: The implant device of any example herein, in particular example 36, wherein the midsection is configured to radially expand into a rupture or tear in a wall of the blood vessel in response to an increase in pressure within the blood vessel and to contract elastically in response to a decrease in pressure within the blood vessel.

Example 39: The implant device of any example herein, in particular example 38, wherein the first anchoring region and the second anchoring region are configured to seal to an inner wall of the blood vessel on opposite ends of the rupture or tear in the wall of the blood vessel.

Example 40: The implant device of any example herein, in particular example 38, wherein the pliant tubular device is configured to form the rupture or tear in the wall of the blood vessel while the pliant tubular device is deployed using a transcatheter procedure.

Example 41: The implant device of any example herein, in particular examples 31-40, wherein the first and second anchoring regions are configured to anchor the implant device within an aorta.

Example 42: The implant device of any example herein, in particular examples 31-41, wherein the implant device is sterilized.

Example 43: A method comprising: advancing an implant device through an anatomic fluid vessel, the implant device including a pliant tubular device having a first anchoring region at a proximal end, a second anchoring region at a distal end, and a flexible midsection disposed therebetween, the first and second anchoring regions being configured to encourage tissue ingrowth at an outer surface of the first and second anchoring regions and the flexible midsection configured to inhibit tissue ingrowth; and deploying the implant device by anchoring at least the first anchoring region to an inner surface of the anatomic fluid vessel.

Example 44: The method of any example herein, in particular example 43, wherein the deploying includes positioning the implant device at a portion of the anatomic fluid vessel having a diminished compliance.

Example 45: The method of any example herein, in particular example 43 or example 44, wherein the deploying includes anchoring the second anchoring region to the inner surface of the anatomic fluid vessel.

Example 46: The method of any example herein, in particular example 43 or example 44, wherein the deploying includes intentionally forming a rupture or a tear in the anatomic fluid vessel at the location of the flexible midsection.

Example 47: The method of any example herein, in particular example 43 or example 44, wherein the deploying includes: positioning the pliant tubular device within the anatomic fluid vessel using a transcatheter procedure, while the pliant tubular device is in a compressed state; expanding the pliant tubular device to the inner surface of the anatomic fluid vessel; waiting a predetermined duration to allow the first and second anchoring regions to bond to the inner surface of the anatomic fluid vessel; and expanding the flexible midsection using an elevated pressure device, to intentionally form a rupture or a tear in the anatomic fluid vessel at the location of the flexible midsection.

Example 48: The method of any example herein, in particular example 46 or example 47, wherein the flexible midsection is configured to radially expand into the rupture or the tear in the anatomic fluid vessel in response to an increase in pressure within the anatomic fluid vessel and to contract elastically in response to a decrease in pressure within the anatomic fluid vessel.

Example 49: The method of any example herein, in particular example 47, wherein the deploying includes bypassing the rupture or the tear with the pliant tubular device.

Example 50: The method of any example herein, in particular example 47, wherein the deploying includes sealing the first anchoring region to the inner surface of the anatomic fluid vessel at a location before the rupture or the tear and sealing the second anchoring region to the inner surface of the anatomic fluid vessel at a location after the rupture or the tear.

Example 51: The method of any example herein, in particular example 50, wherein the sealing of the first and second anchoring regions to the inner surface of the anatomic fluid vessel seals the first and second anchoring regions against fluid leakage from between the first or second anchoring regions and the inner surface of the anatomic fluid vessel.

Example 52: The method of any example herein, in particular example 43, wherein the deploying includes engaging a first anchoring structure associated with the first anchoring region to the inner surface of the anatomic fluid vessel.

Example 53: The method of any example herein, in particular example 52, wherein the deploying includes engaging a second anchoring structure associated with the second anchoring region to the inner surface of the anatomic fluid vessel.

Example 54: The method of any example herein, in particular example 53, wherein at least one of the first anchoring structure or the second anchoring structure comprises an expandable stent, a docking station for a prosthetic valve, or a prosthetic valve.

Example 55: The method of any example herein, in particular example 53, wherein at least one of the first anchoring structure or the second anchoring structure includes shape-memory metal.

Example 56: The method of any example herein, in particular example 53, wherein at least one of the first anchoring structure or the second anchoring structure comprises an anchor or a pin configured to anchor the implant device to the inner surface of the anatomic fluid vessel.

Example 57: The method of any example herein, in particular example 43, wherein a material of the flexible midsection is different from a material of the first anchoring region or the second anchoring region.

Example 58: The method of any example herein, in particular example 43, wherein the implant device includes a structural form disposed within the pliant tubular device configured to prevent the pliant tubular device from radially compressing.

Example 59: The method of any example herein, in particular examples 43-58, wherein the anatomic fluid vessel is an aorta.

Example 60: The method of any example herein, in particular examples 43-59, wherein the implant device is sterilized.

Depending on the example, certain acts, events, or functions of any of the processes or algorithms described herein can be performed in a different sequence, can be added, merged, and/or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without author input or prompting, whether these features, elements, and/or steps are included or are to be performed in any particular example. The terms “comprising,” “including,” “having,” and the like are generally synonymous, used in their ordinary sense, and used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is understood with the context as used in general to convey that an item, term, element, etc. can be either X, Y, or Z. Thus, such conjunctive language is not generally intended to imply that certain examples require at least one of X, at least one of Y, and at least one of Z to each be present.

In examples, various features are sometimes grouped together in a single example, figure, or description thereof for the purpose of streamlining the disclosure and aiding in the understanding of one or more of the various aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the subject matter herein disclosed and claimed below should not be limited by the particular examples described herein.

Certain ordinal terms (e.g., “first” or “second”) may be provided for case of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather can generally distinguish the element from another element having a similar or identical name (but for use of the ordinal term). In addition, as used herein, indefinite articles (“a” and “an”) can indicate “one or more” rather than “one.” Further, an operation performed “based on” a condition or event can also be performed based on one or more other conditions or events not explicitly recited.

Unless otherwise defined, terms (including technical and/or scientific terms) used herein can have the same meaning as commonly understood by one of ordinary skill in the art to which examples belong. Terms, such as those defined in commonly used dictionaries, can be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, can be used herein for ease of description to describe the relations between one element or component and another element or component as illustrated in the drawings. Spatially relative terms can encompass different orientations of the device in use or operation, in addition to the orientation depicted in the drawings. For example, in the case where a device shown in the drawing is turned over, the device positioned “below” or “beneath” another device can be placed “above” another device. Accordingly, the illustrative term “below” can include both the lower and upper positions. The device can also be oriented in the other direction, and thus the spatially relative terms can be interpreted differently depending on the orientations.

Unless otherwise expressly stated, comparative and/or quantitative terms, such as “less,” “more,” “greater,” and the like, can encompass the concepts of equality. For example, “less” can mean not only “less” in the strictest mathematical sense, but also “less than or equal to.”

What is claimed is:

1. An implant device comprising:
a pliant tube including a first end and a second end, including:
a first anchoring region disposed at the first end;
a second anchoring region disposed at the second end, the first anchoring region and the second anchoring region configured to anchor the implant device within an anatomical structure and to encourage tissue ingrowth at an outer surface of the first anchoring region and an outer surface of the second anchoring region; and
a flexible midsection disposed between the first anchoring region and the second anchoring region, the midsection comprising a different material from a material of the first and second anchoring regions.

2. The implant device of claim 1, wherein the implant device is configured to extend within a segment of a blood vessel.

3. The implant device of claim 1, further comprising a first anchoring structure associated with the first anchoring region, configured to anchor the first anchoring region to an inner surface of the anatomical structure.

4. The implant device of claim 3, wherein the first anchoring structure comprises an expandable stent.

5. The implant device of claim 3, wherein the first anchoring structure comprises a prosthetic valve or a docking device for a prosthetic valve.

6. The implant device of claim 1, wherein at least one of the first and second anchoring regions is comprised of a natural or synthetic fabric that is coated with a compound that encourages tissue ingrowth.

7. The implant device of claim 6, wherein an inner surface of the first and second anchoring regions is free from the compound that encourages tissue ingrowth.

8. The implant device of claim 6, wherein the natural or synthetic fabric is disposed over an expandable stent.

9. The implant device of claim 1, wherein at least one of the first and second anchoring regions is comprised of a natural or synthetic fabric that is fabricated from a compound that encourages tissue ingrowth.

10. The implant device of claim 1, wherein the midsection is configured to radially expand in response to an increase in pressure within the anatomical structure and to contract elastically in response to a decrease in pressure within the anatomical structure.

11. The implant device of claim 1, wherein the midsection is fabricated from a compound that inhibits tissue ingrowth or is coated with a compound that inhibits tissue ingrowth.

12. An implant device comprising:
a tubular graft having a proximal end and a distal end;
a first anchoring region disposed at the proximal end, configured to encourage tissue ingrowth at an outer surface of the first anchoring region;
a second anchoring region disposed at the distal end, configured to encourage tissue ingrowth at an outer surface of the second anchoring region; and
a flexible midsection disposed between the proximal end and the distal end, configured to inhibit tissue ingrowth at the flexible midsection.

13. The implant device of claim 12, wherein at least one of the first anchoring region and the second anchoring region includes an anchoring structure configured to anchor the implant device within a segment of a blood vessel.

14. The implant device of claim 12, wherein the first anchoring region includes a first expandable stent configured to anchor the implant device to an inner surface of an anatomical structure.

15. The implant device of claim 13, wherein the anchoring structure includes a first natural or synthetic tissue that is coated at an outer surface of the first natural or synthetic tissue with a compound that encourages tissue ingrowth.

16. The implant device of claim 13, wherein the anchoring structure includes a first natural or synthetic tissue that is fabricated from a compound that encourages tissue ingrowth or is coated with a compound that inhibits tissue ingrowth.

17. The implant device of claim 12, wherein the first anchoring region and the second anchoring region are configured to seal to an inner wall of an anatomical structure to prevent fluid leakage from between the first anchoring region and the inner wall of the anatomical structure and the second anchoring region and the inner wall of the anatomical structure.

18. The implant device of claim 12, wherein the midsection is comprised of a thromboresistant material or is coated with a thromboresistant material.

19. An implant device comprising:
a pliant tubular device configured to be implanted within an anatomical structure, having a first anchoring region at a proximal end, a second anchoring region at a distal end, and a flexible midsection disposed therebetween, the first and second anchoring regions being configured to encourage tissue ingrowth at an outer surface of the first and second anchoring regions and the flexible midsection configured to inhibit tissue ingrowth at an outer surface and an inner surface of the flexible midsection.

20. The implant device of claim 19, wherein the pliant tubular device is configured to be implanted within a segment of a blood vessel, and wherein the midsection is configured to radially expand into a rupture or tear in a wall of the blood vessel in response to an increase in pressure within the blood vessel and to contract elastically in response to a decrease in pressure within the blood vessel.

21. The implant device of claim 20, wherein the first anchoring region and the second anchoring region are configured to seal to an inner wall of the blood vessel on opposite ends of the rupture or tear in the wall of the blood vessel.

22. The implant device of claim 20, wherein the midsection of the pliant tubular device is configured to radially expand to form the rupture or tear in the wall of the blood vessel while the pliant tubular device is deployed using a transcatheter procedure.

23. The implant device of claim 19, wherein the implant device is sterilized.
